(19) United States
(12) Patent Application Publication
(10) Pub. No.: US 2026/0244639 A1
(43) Pub. Date: Aug. 20, 2026

(54) SYSTEMS AND METHODS FOR INGREDIENT-PRODUCT LINKING AND UNIT CONVERSION

(71) Applicant: Direct Supply, Inc., Milwaukee, WI (US)

(72) Inventors: Michael Conner, Brookfield, WI (US); Sam Keyser, Milwaukee, WI (US); Meagan E. Harrison, Lakewood, WI (US); Luke Harwood, Waukesha, WI (US); Alexander Neuwirth, Milwaukee, WI (US); Benjamin Paulson, Milwaukee, WI (US); Robert Burkhardt, Milwaukee, WI (US); Ezra McNichols, Greendale, WI (US); Andrew Schwabe, Racine, WI (US)

(21) Appl. No.: 19/541,615

(22) Filed: Feb. 17, 2026

Related U.S. Application Data
(60) Provisional application No. 63/759,509, filed on Feb. 17, 2025.

(51) Int. Cl. G06F 16/25; G06F 16/2457; G06F 16/248

(52) U.S. Cl. CPC G06F 16/258; G06F 16/24575; G06F 16/248

(57) ABSTRACT

The system for menu optimization utilizes various models and modules to streamline the process of menu planning and management. It incorporates an ingredient-product linking model, a unit conversion model, a nutritional module, and an optimizations model to process menu data and generate recommendations. The system may determine replacement recipes based on nutritional similarity, recipe affinity, and tag-based filtering, while considering costs. By leveraging these components, the system may provide efficient and accurate menu recommendations that balance nutritional requirements, dietary restrictions, cost considerations, and ingredient availability.

### CROSS REFERENCE TO RELATED APPLICATION

This application claims priority to and the benefit of U.S. Provisional Patent Application No. 63/759,509, filed Feb. 17, 2025, the contents of which is incorporated herein by reference in its entirety.

### BACKGROUND

Menu planning and management in industries such as healthcare, senior living, and restaurants often involve complex processes that can be time-consuming and inefficient. Traditional methods for menu optimization may not adequately account for factors like nutritional requirements, dietary restrictions, cost considerations, and ingredient availability. Existing systems struggle to provide accurate and timely recommendations for menu adjustments that balance nutritional needs, cost constraints, and resident preferences.

### BRIEF DESCRIPTION OF THE DRAWINGS

FIG. 1 illustrates a diagram of menu system, according to some embodiments of the present technology.

FIG. 2 illustrates a diagram of a process for linking an ingredient to a product, according to some embodiments of the present technology.

FIG. 3 illustrates a diagram of a process for product to recipe ingredient conversions, according to some embodiments of the present technology.

FIG. 4 illustrates a diagram of a process for a conversion determined using embedding table lookup, according to some embodiments of the present technology.

FIG. 5 illustrates a diagram of a process for unit conversion, according to some embodiments of the present technology.

FIG. 6 illustrates a diagram of a unit conversion database architecture and ingestion pipeline, according to some embodiments of the present technology.

FIG. 7A illustrates a flow diagram illustrating a method of automating product ordering based on menus, according to some embodiments of the present technology.

FIG. 7B illustrates a meal count user interface configured in accordance with some embodiments of the present technology.

FIG. 7C illustrates a purchasing guide user interface configured in accordance with some embodiments of the present technology.

FIG. 7D illustrates a user interface for preparing a menu order configured in accordance with some embodiments of the present technology.

FIG. 7E illustrates a user interface for a purchasing guide configured in accordance with some embodiments of the present technology.

FIG. 7F illustrates a user interface for preparing a menu order and managing inventory configured in accordance with some embodiments of the present technology.

FIG. 7G illustrates a user interface for managing staples configured in accordance with some embodiments of the present technology.

FIG. 8A illustrates a diagram for a product to ingredient unit conversion process, according to some embodiments of the present technology.

FIG. 8B illustrates a unit conversion search interface configured in accordance with some embodiments of the present technology.

FIG. 8C illustrates an override user interface configured in accordance with some embodiments of the present technology.

FIG. 9 illustrates a diagram of a recommendation model, according to some embodiments of the present technology.

FIG. 10 illustrates a diagram of a process for generating candidate recipes, according to some embodiments of the present technology.

FIG. 11 illustrates a diagram of a process for constructing a network graph, according to some embodiments of the present technology.

FIG. 12 illustrates a diagram of a process for identifying candidate recipes, according to some embodiments of the present technology.

FIG. 13 illustrates a diagram of an example graph showing a relationship between two entrees connected by two sides, according to some embodiments of the present technology.

FIG. 14 illustrates a candidate recipe scoring graph, according to some embodiments of the present technology.

FIGS. 15-18 illustrate examples of menu user interfaces, according to some embodiments of the present technology.

FIG. 19 illustrates an example of a recipe shown in a user interface, according to some embodiments of the present technology.

FIG. 20 illustrates an example of individual ingredient details displayed in a user interface, according to some embodiments of the present technology.

FIG. 21 is a flow diagram illustrating an example process of updating a recipe, according to some embodiments of the present technology.

FIG. 22 is a block diagram that illustrates an example AI system that can implement aspects of the present technology.

FIG. 23 illustrates a user interface for displaying resident information configured in accordance with various embodiments of the present technology.

FIG. 24 illustrates a user interface for recipe swap recommendations configured in accordance with various embodiments of the present technology.

FIG. 25 illustrates a user interface for updated recipe details configured in accordance with various embodiments of the present technology.

FIG. 26 illustrates a user interface for overriding product unit conversions configured in accordance with various embodiments of the present technology.

FIGS. 27A-27B illustrate a user interface for an enhanced ingredient view configured in accordance with various embodiments of the present technology.

FIG. 28 illustrates a user interface for setting individual recipe forecast percentages configured in accordance with various embodiments of the present technology.

FIG. 29 illustrates a user interface for setting servings per meal at a location level configured in accordance with various embodiments of the present technology.

FIGS. 30A-30B illustrate a user interface for nutritional analysis configured in accordance with various embodiments of the present technology.

FIG. 31 illustrates a user interface for a purchasing workflow configured in accordance with various embodiments of the present technology.

FIGS. 32A-32B illustrate a user interface for menu views across diet types configured in accordance with various embodiments of the present technology.

FIG. 33 is a block diagram that illustrates an example of a computer system in which at least some operations described herein can be implemented.

The headings provided herein are for convenience only and do not necessarily affect the scope of the embodiments. Further, the drawings have not necessarily been drawn to scale. For example, the dimensions of some of the elements in the figures may be expanded or reduced to help improve the understanding of the embodiments. Moreover, while the disclosed technology is amenable to various modifications and alternative forms, specific embodiments have been shown by way of example in the drawings and are described in detail below. The embodiments are intended to cover all suitable modifications, combinations, equivalents, and alternatives falling within the scope of this disclosure.

### DETAILED DESCRIPTION

Various examples of the systems and methods introduced above will now be described in further detail. The following description provides specific details for a thorough understanding and enabling description of these examples. One skilled in the relevant art will understand, however, that the techniques and technology discussed herein may be practiced without many of these details. Likewise, one skilled in the relevant art will also understand that the technology can include many other features not described in detail herein. Additionally, some well-known structures or functions may not be shown or described in detail below so as to avoid unnecessarily obscuring the relevant description.

The present technology relates to an AI-driven system designed to optimize menu planning and management for various industries such as healthcare, senior living, and restaurants. It provides features such as automated menu costing, product ordering, ingredient substitution recommendations, and nutritional analysis. The system can integrate with third-party vendors and production systems and supports procurement and inventory management. The system uses AI models to link ingredients to vendor products and perform different types of complex unit conversions for determining accurate quantities of product to order, accurate recipe cost and nutritional analysis per serving/resident. The system determines recipe similarity for recommending cost-optimized nutritionally equivalent recipes. The system also considers dietary restrictions, regional preferences, regulatory compliance, and industry-specific needs, providing a comprehensive solution for efficient and cost-effective menu management. As such, the system can make recommendations for substitute or alternative recipes that differ based on various factors such as preferences, dietary types, dietary restrictions, etc.

By effectively mapping ingredients to vendor products and handling complex conversions, this system provides efficient and accurate menu management, ensuring both cost-effectiveness and nutritional accuracy. The menu system includes several independent workflows/processes/models to accomplish the desired outcomes of: 1) ingredient-product linking and quantity conversions; and 2) recipe optimization recommendations (e.g., recipe nutritional similarity, recipe affinity, and recipe tag filtering).

The present technology provides a hybrid menu planning and optimization tool. The tool can streamline identifying and changing out recipes to reduce cost while maintaining nutritional coverage and recipe affinity. This is accomplished by using nutritional similarity, recipe affinity, and tag filtering to identify recipes. The tool uses a unit conversion model to align formularies to menu selections, determine the amount of product to order, determine the cost of individual recipes to provide one-to-one comparisons, identify substitute recipes to cover therapeutic diets, and identify and replace ingredients to modify recipes to achieve further optimization and coverage.

Methods and systems disclosed herein can provide technical advantages over conventional systems. The present technology provides a tool for complex product to recipe ingredient unit conversions and makes use of LLM, to: 1) properly calculate recipe cost and nutritionals for optimization recommendations; 2) use a menu to drive product ordering, and specifically recommended order quantities; 3) synchronize formularies to menu selections; 4) improve scalability; 5) identify substitute recipes for therapeutic diet coverage; 6) substitute ingredients for cost savings, and dynamically modify a recipe if needed; and 7) provide calendar view for hybrid menu planning and optimization identifications.

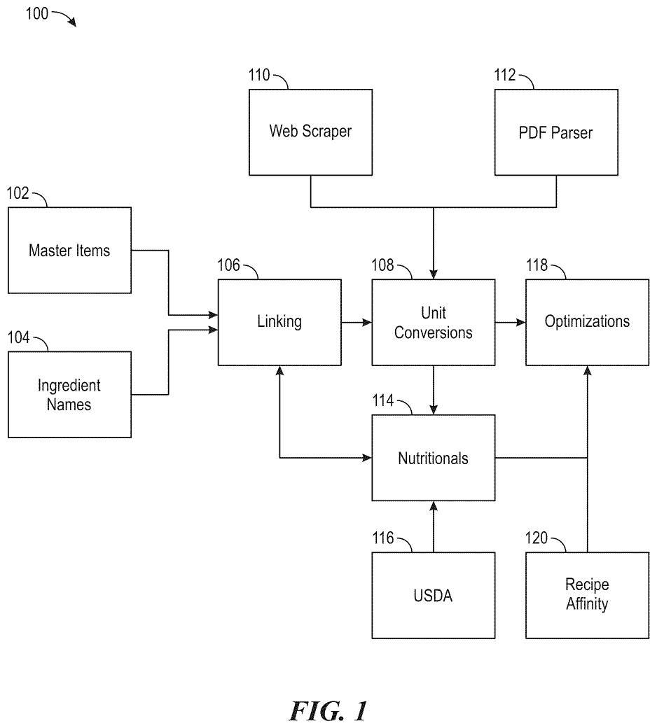

FIG. 1 illustrates a diagram 100 of a menu system. Diagram 100 illustrates the interaction of AI components of the menu system. Master items 102 and ingredient names 104 are linked together using an ingredient-product linking model 106, which uses embeddings to determine an appropriate master item and thus product(s) for a particular recipe ingredient. A unit conversion model 108 is used to select the appropriate conversion from the product units to the recipe ingredient units, including accounting for the edible portion (i.e., loss) of the as-purchased product. The unit conversions are obtained from web scraper module 110 and PDF parsing module 112 but can also be manually entered. A nutritional module 114 pulls/identifies appropriate nutritional information for ingredients using product nutrition information and USDA nutrition information when not known from product information from USDA module 116. This can require a unit conversion specific to nutritional to determine the nutritional amounts (e.g., USDA weight equivalents) per serving of a recipe. An optimizations model 118 is used for determining replacement recipes based on a combination of nutritional similarity, recipe affinity similarity, and tag-based recipe filtering. The replacement recipes can then be surfaced or prioritized based on appropriate per portion prices of the recipe using per portion prices of ingredients from the identified product from product linking, and unit conversions from product to ingredient. The recipe affinity model 120 determines appropriate replacement recipes based on similar co-occurrence with other recipes in meals in a menu.

Ingredient-product linking and quantity conversion is a two-part process that (1) maps ingredients to products and (2) performs unit conversions including converting the product units to appropriate recipe units and recipe ingredient units to nutritional units (e.g., USDA weight equivalent units). The second conversion from recipe ingredient units to nutritional units may be independent of the product linked to the ingredient. This system is essential for accurately costing recipes, determining accurate order quantities, and analyzing nutritional macros and micros of recipes for menu regulation compliance and determining appropriate recommendations for substitute or alternate recipes.

Example uses of the ingredient-product linking and quantity conversion modules can include: 1) determining order quantities, such as determining the precise amount of each ingredient needed for the recipes to serve the residents of a facility, which can be used to determine the appropriate amount of each product to order by converting the product units to recipe ingredient units; 2) costing recipes to ensure accurate pricing by linking ingredients to corresponding vendor products and converting the price per product unit to price per ingredient portion; and 3) nutritional analysis by calculating the nutritional content of recipes based on the linked product to ingredients and converting recipe ingredient units to USDA weight units. The modules can be used for menu regulatory compliance and determining nutritionally equivalent recipes and alternative recipes for different diet types.

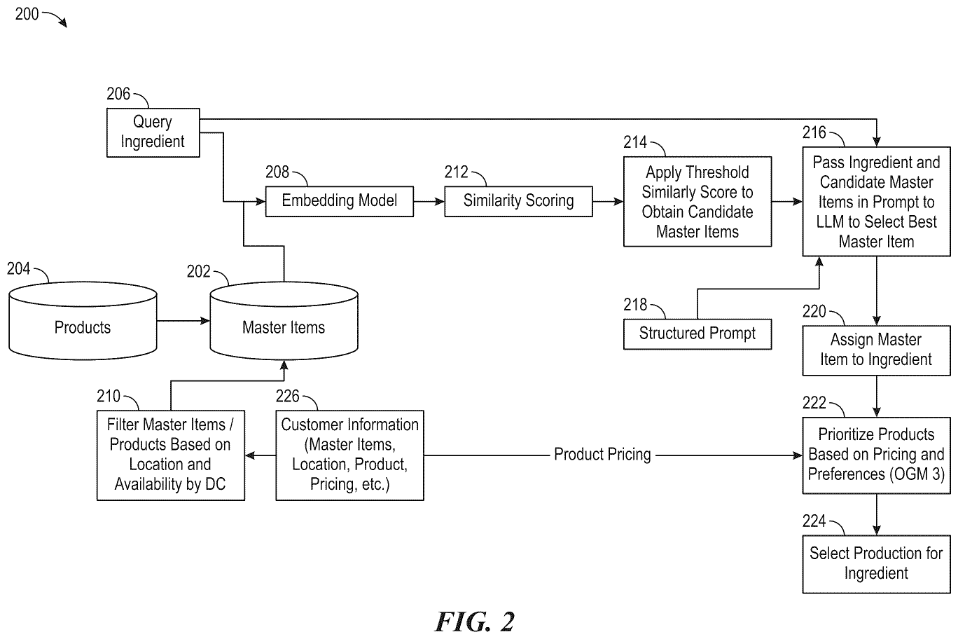

FIG. 2 illustrates a diagram of a process 200 for linking an ingredient to a product, according to some embodiments of the present technology. The ingredients are linked to a master item and therefore the appropriate products for a facility (i.e., products that a facility can order and be fulfilled from a distribution center and are not out-of-stock or discontinued).

At step 206, process 200 includes embedding model 208 receiving a query ingredient that needs an appropriate master item 202 and corresponding products 204 identified. At step 210, process 200 includes retrieving potential master items based on the customer (e.g., a provider). Master item and product availability can be filtered/retrieved based on customer location and distribution center (DC). For example, each facility may have different master items and corresponding different product availability based on their covering distribution center.

The embedding model 208 can generate embeddings. One or more equivalent master items for the ingredient can be determined based on similarity of an embedding associated to the ingredient and an embedding associated with potential master items. The embedding for the ingredient is generated using the ingredient name. The ingredient name is preprocessed to identify and remove verbs (e.g., chopped, diced). The master item embeddings are generated for each master item from a combination of the master item name, combined with the names and descriptions of all the products within the master item.

Embedding model 208 can perform preprocessing of the ingredient information, such as reformatting or expanding abbreviations (e.g., expanding SF to sugar free). The embedding model 208 can be OpenAI Text-embedding-3 combined with a fine-tuned deep neural network with circle loss based on user feedback.

At step 212, process 200 includes calculating similarity scores. For each ingredient to master item pair, process 200 includes calculating a similarity score based on the embeddings (i.e., distance between the two embeddings) using cosine similarity. At step 214, process 200 includes applying a threshold similarity score to the calculated similarity scores to filter out master items and obtain a list of potential candidate master items. For example, selecting the top 5 scores that are above 80% similarity 5. At step 216, process 200 includes passing the list of candidate master items for the ingredient to a LLM. The LLM is requested in a structured prompt 218 to select the master item that is the best possible match to the ingredient. For example, the information provided to the LLM in the prompt includes:

| | |
|---|---|
| □ | request (e.g., via an XML output and that is parsed), such as: |
| | { |
| | “Name”: “fill_ingredient_with_product”, |
| | “description”: ″Get the price for an ingredient using the |
| | selected product group and product″, |
| | “required_parameters”: [“product_group”], |
| | “parameters”: [ |
| | { |
| | “name”: “product_group”, |
| | “type”: “enum”, |
| | “enumType”: “string” |
| | “enum”: [this is filled with the candidate master |
| | items dynamically] |
| | } |
| | ] |
| | } |
| □ | passed a prompt of: |
| | “””I am pricing a {insert ingredient name} I have the following |
| | product groups:\nGroup: (master item name)\n- product name |
| | / description in a list\n Which product group is the most similar |
| | to the ingredient? |
| | ””” |
| □ | ingredient Name |
| □ | a list of each candidate master item (master item name) each with a |
| | corresponding list of available products (product name and description). |

At step 220, process 200 includes assigning a master item to an ingredient. At step 222, process 200 includes prioritizing products based on pricing and preferences, such as OGM3 product prioritization. For example, the products of the assigned master item can be prioritized based on product pricing, normalizing the pricing to base units for comparison (i.e., to account for products sold in different unit and pack sizes). At step 224, the top priority product is selected for an ingredient unless overridden by customer preferences or other factors. At step 226, product pricing is pulled for the particular customer based on stored pricing from contracts. Note this includes filtering based on facility location and what products are available at the facility based on availability at the corresponding distribution center. It can also account for inventory level availability at the distribution center.

The master items may be the same for all facilities/locations associated with a customer (although they may also differ based on facility/location). Product availability can be dependent on the facility location and availability at the corresponding distribution center(s). Accordingly, an ingredient for a recipe may be linked to multiple products (e.g., via the master item) to ensure coverage of all facilities. This may warrant the addition of a product to an order guide for one facility even if another product for that ingredient already exists but is available for another facility.

Individual product pricing is dependent and pulled for each customer and location, such as a pricing and service API. Every customer can have a different contract with different individual product pricing and is updated based on data feeds from suppliers. Pricing can also take into account temporary price adjustments such as rebatements.

Products are prioritized based on normalized pricing and/or other factors, such as customer configurations and preferences. The normalized price is the price per base-unit (i.e., a common standard unit of measurement) of each product needs to be determined to be able to do a one-to-one comparison. Often, products differ in their base units (e.g., product A is 12 oz each sold in packs of 12, while product B is 14 oz each sold in packs of 10) such that the price of the product cannot be directly compared without determining the price per a common unit (e.g., price per ounce, price per each, etc.).

Product to recipe ingredient unit conversion and recipe ingredient to nutritionals, unit conversion are two types of conversions that are done, and are used for different purposes. The product to recipe ingredient unit conversion process takes the units and unit of measure of the as purchased product and converts to the units and unit of measure of the ingredient of the recipe to the ingredient units and unit of measure. This process can account for the edible portion percentage of a product (i.e., how much of the as-purchased product by weight/volume can actually be used to account for the required amount of the ingredient in the recipe). This conversion is important in determining how much of a product needs to be purchased to make enough servings (aka portions) of a recipe for the residents at the facility (that will be eating the recipe). The per serving ingredient cost for a recipe can be combined in pricing the per resident/serving cost of each recipe. This recipe pricing is useful to be able to provide recipe price optimizations.

The recipe ingredient to nutritionals, unit conversion process takes the units of the recipe ingredient for one portion of the recipe and calculates the nutritionals, such as macro and micro-nutrients, or USDA weight units (i.e., ounce equivalents of the USDA My Plate Category: protein, grains, fruits, vegetables, dairy). This process is important for calculating the amount of nutritionals provided by each serving of individual ingredients and corresponding recipes. These per serving recipe nutritional are used in automating nutritional summaries (for meals, days, etc.) and providing recipe nutritional optimizations (i.e., recipe equivalents based on nutritionals).

In some embodiments, the embedding model 208 may utilize efficient similarity search structures such as approximate nearest neighbor indexes to perform rapid vector comparisons across large collections of master item embeddings. For example, the system may employ a Hierarchical Navigable Small World (HNSW) index for vector search, which enables scalable similarity searches even when the number of master items and associated products is substantial. The HNSW index structure allows the system to quickly identify candidate master items without exhaustively comparing the query ingredient embedding against every master item embedding in the database, thereby reducing computational overhead while maintaining search accuracy.

The LLM at step 216 may be configured to return responses in a structured format such as XML, which the system parses to extract the selected master item. In some aspects, the structured prompt 218 may incorporate few-shot learning by including example ingredient-to-master-item mappings within the prompt to guide the LLM's selection process. The examples provided in the prompt may demonstrate correct matching patterns, helping the LLM understand the desired output format and selection criteria. Additionally, an ingredient may be linked to multiple master items in various scenarios, such as when different organizations have distinct product catalogs, when different order guides specify alternative products, or when different distribution centers stock different product variants. This multi-master-item linking allows the system to accommodate the varying product availability and procurement arrangements across different facilities and customer configurations.

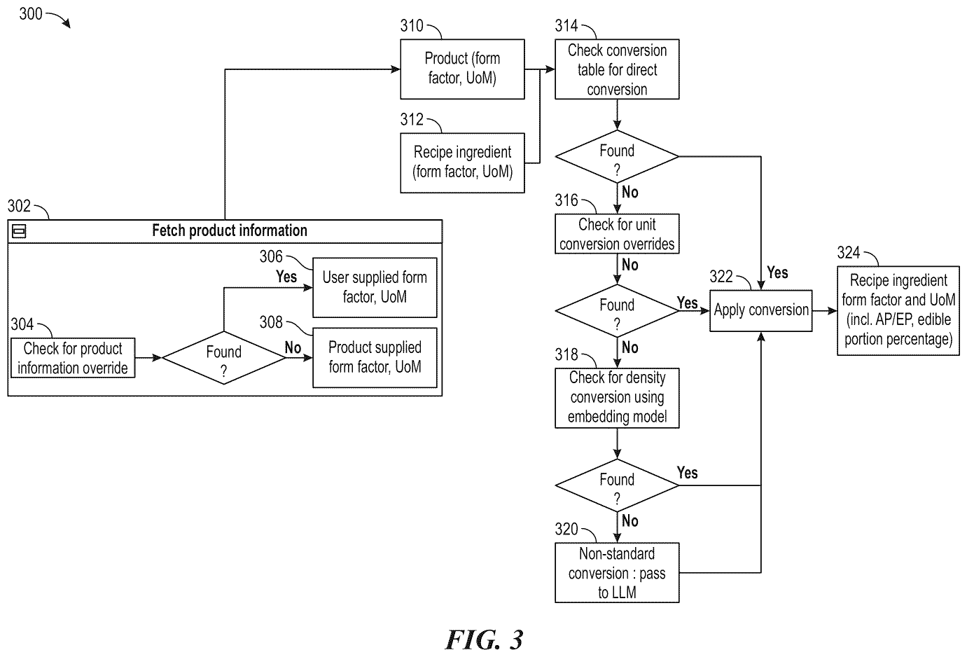

FIG. 3 illustrates a diagram of a process 300 for product to recipe ingredient conversions, according to some embodiments of the present technology. The units and unit of measure of the product can be converted to the units and the unit of measure of the ingredient required by the recipe. The conversion process involves four tiers of complexity: 1) direct conversions; 2) user-sourced conversions; 3) indirect (yield table) conversions; and 4) generated/hallucinated conversions.

Direct conversions involve conversions between different units in the same form factors. The form factors that can be converted can include quantity, weight (or mass), and volume. Each form factor has a standard unit of measure: count for quantity, ounces for weight, and cups for volume. Direct conversion is a list of conversion factors to convert between units of measure. An example of a direct conversion is mass to mass (e.g., grams to ounces) or volume to volume (e.g., cups to liters). The table can also separately store AP/EP (i.e., percent of the as-purchased quantity that is edible, used for the ingredient, as described further below).

Indirect (yield table) conversions handle more complex conversions between different form factors (e.g., weight to volume, weight to count, volume to count, etc.). These can require understanding the density of ingredients. For example, converting flour from mass to volume (e.g., ounces to cups). As another example, converting uncooked rice from mass to volume (e.g., grams to cups). The density can be an estimate, such as converting quantity of a particular fruit/vegetable; quantity of whole medium onions to volume (cups) of onions.

The yield table conversions can also take into account the processing step (e.g., mashed, chopped, etc.) and other metadata (e.g., size categories, such as small, medium, and large) for specific conversions. For example, converting from quantity (count) of whole bananas to volume (cups) of chopped bananas. As mentioned, above, some conversions incorporate AP/EP, which is accounts for the edible portion or percentage of the as-purchased product by volume/weight that is used for the ingredient by the recipe (subtracting for a used portion) as discussed further below. The AP/EP may be stored as a separate value or simply accounted for in the conversion.

Embeddings are used to identify the most likely indirect conversions from a lookup table to apply. In version 1, discussed below, this uses product and ingredient names/descriptions and units. As described below, in version 2, only the product is used for the embedding to identify a set of conversions, then the form factor of the ingredient is used to determine the specific conversion appropriate for the ingredient.

Generated/hallucinated conversions include conversions not in the yield table. This includes conversions such as converting leaves of lettuce or cloves of garlic. These conversions often require reasoning through the specifics of the ingredient, sometimes involving a language model to determine the best approach. Conversions may be cached in the yield table for future use.

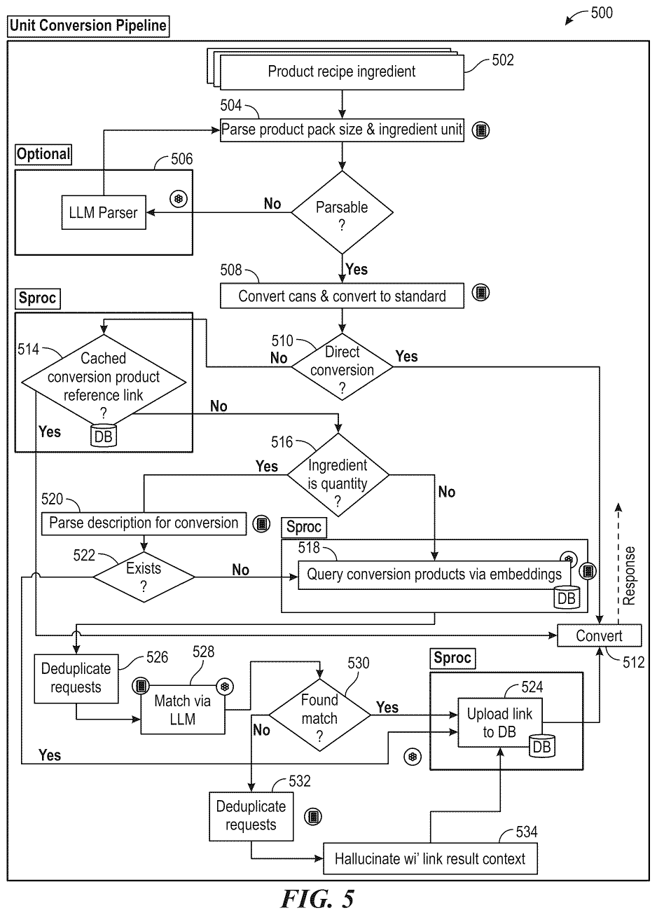

FIG. 3 shows a process for selecting each of the different conversions. As shown in FIG. 5, the product may need to first be converted to “each” (i.e., an individual quantity), if the ingredient is each, but the product is not. If an each-conversion is not cached for the particular product, a LLM model may be queried using the product name and description with a specific prompt to determine a conversion for the product.

For example, product quantities that are expressed in denominations of a particular can size (e.g., #10 can) are also converted to a volume (e.g., 12 cups), based on a list of can size to volume conversions. Due to canned volume of food being inaccurate (e.g., from water content), a warning may be indicated in a UI to the end user that the conversion may be off for canned foods.

Before performing the direct conversion, the unit of measure for the product is also converted to standard units using a direct conversion, such as weight to ounces, volume to cups, and quantity to count (i.e., “each”, as described above).

With continued reference to FIG. 3, the process 300 begins at step 302, where the process 300 includes collecting the product form factor and unit of measure (UoM). At step 304, the process 300 includes collecting the recipe ingredient form factor and unit of measure (UoM).

At step 306, the process 300 includes checking for a product information override. At step 308, the process 300 determines whether a product override (e.g., product pack size override) is found. If a product override is found (Yes branch), the process 300 uses the user-supplied form factor and UoM. If no product override is found (No branch), the process 300 uses the product-supplied form factor and UoM. The process 300 then fetches product information at a step 310, which includes the product form factor and UoM.

At a step 312, the process 300 collects the recipe ingredient form factor and UoM. The process 300 proceeds to a step 314, where the system checks the conversion table for a direct conversion. At a step 316, the process 300 determines whether a direct conversion is found. If a direct conversion is found (Yes branch from step 316), the process 300 proceeds to a step 324 to apply the conversion.

If no direct conversion is found (No branch from step 316), the process 300 moves to a step 318 to check for unit conversion overrides. If a unit conversion override is found (Yes branch from step 318), the process 300 proceeds to step 324 to apply the conversion. If no unit conversion override is found (No branch from step 318), the process 300 proceeds to a step 320 to check for a density conversion using an embedding model. If a density conversion is found (Yes branch from step 320), the process 300 proceeds to step 324 to apply the conversion.

If no density conversion is found (No branch from step 320), the process 300 moves to a step 322, which handles non-standard conversion by passing the conversion request to a large language model (LLM). The LLM can synthesize a conversion using top conversions based on similarity score above a set threshold (e.g., 80%) as context. The process 300 then proceeds to step 324 to apply the generated conversion.

At step 324, the process 300 applies the conversion. The output at step 324 includes the recipe ingredient form factor and UoM, including the as-purchased to edible portion (AP/EP) percentage. The edible portion percentage (AP/EP factor) accounts for how much of the as-purchased product by weight or volume can actually be used to account for the required amount of the ingredient in the recipe. For example, the weight of bananas as purchased (whole) is more than when the bananas are peeled and chopped for a recipe. Similarly, the AP/EP factor accounts for steps like dicing bell peppers or cracking eggs, where a percentage of food product is not usable as the ingredient and thus cannot be counted towards recipe ingredient requirements. A weight (ounces) of a product “whole pepper” when converted to a volume (cups) of “diced pepper” can account for an estimated loss from a portion of the “whole pepper” that is discarded.

The AP/EP factor may be applied as a separate conversion factor by multiplying the converted units by the AP/EP factor. Alternatively, the AP/EP factor may be a separate conversion step that reduces the as-purchased amount units by a certain percentage after converting to the ingredient units (e.g., 1 unit as-purchased has 0.8 units edible-portion for a particular product-ingredient-conversion). One product may have no reduction due to an edible portion when converted to be used for one ingredient but may have a reduction when converted to be used for another ingredient (e.g., ingredient A=whole-peppers with AP/EP=1; ingredient B=diced peppers with AP/EP=0.8). The edible portion (AP/EP) factor is useful in accurately determining how much product to order and for calculating per patient/serving recipe cost.

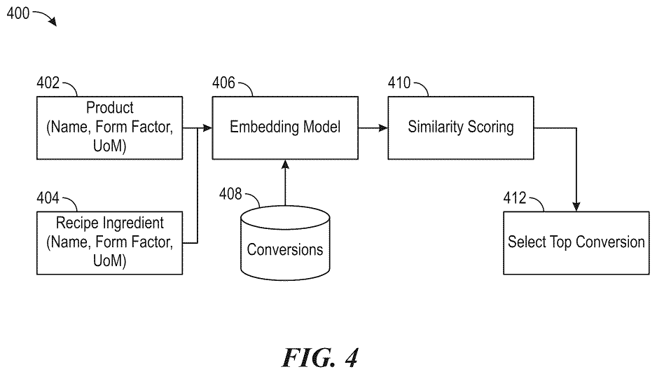

FIG. 4 illustrates a diagram of a process 400 for a conversion determined using embedding table lookup, according to some embodiments of the present technology.

At step 402, process 400 includes collecting the product form factor and unit of measure (UoM). At step 404, process 400 includes collecting the recipe ingredient form factor and unit of measure (UoM). At step 406, process 400 includes generate embeddings with an embedding model. The embedding model can query product name and a yield table, such as by key name of yield table entry (e.g., “Raw Chicken Breast”; “Chopped Red Bell Pepper”).

At step 410, process 400 includes calculating a similarity score between query embedding and conversion embeddings from the Yield Table of conversions 408. (e.g., cosine similarity). At step 412, process 400 includes selecting a top match conversion. For example, taking the top 5 with above 80% match, then passing the selections to LLM to select best based on a similarity score above a threshold. This includes the necessary information for performing the unit conversion. The process 400 can collecting a set of product to ingredient conversion form factors (i.e., weight, volume, or quantity) with corresponding UoM's.

Process 400 can include generating (“hallucinate”) a conversion using an LLM. If no suitable yield-table conversion match is found in (2), the system then identifies it as a non-standard conversion and uses a language model (LLM) to reason through (“hallucinate”) the specifics of the ingredient and conversion. The results are then parsed to determine how to apply the conversion, the prompt specifies how the conversion output should be formatted to allow parsing. The prompt includes: 1) product name; 2) product pack size (e.g. 2/30 oz, 2 items of 30 oz each); 3) top-5 similar conversion records (e.g., salt, 16 oz=12 floz); 4) target unit (e.g. volume); 5) valid units (e.g. weight: oz); 6) LLM output format as shown in Table 1.

### Prompt:

You are a unit conversion expert that must use domain knowledge to determine how to convert a product to the target units. You will be given a list of products that are similar to the product we want to convert. You will also be given the product, the amount the product is sold in, and the target units. You must give a conversion that will go straight from the product amount to the target unit type given the available units. Even if there aren't similar products, you must still give a conversion. Think critically about the product and the available conversions and perform minimal calculations. Your answer must consist of a well formatted conversion with no additional text.
Follow the following format very carefully:

### Input:

<product>[the name of the product we want to convert]</product>
<amount>[the amount the product is sold in]</amount>
<similarProducts>
[a list of products that are similar, along with their conversions]
</similarProducts>
<targetUnit>[the target unit of measure]</targetUnit>

### Output:

<reasoning>[reason your answer, think critically]</reasoning>
<conversion>[conversion in the format number unit=number unit]</conversion>

### Example:

```
INPUT:
<product>SALT PACKET .6 GM</product>
<amount>6/1000CT</amount>
<similarProducts>
Salt
  16.0 oz = 12.0 floz - (conversion was given for 1 1/2 cups)
  1.0 oz = 0.75 floz - (conversion was given for 1 1/2 Tbsp)
Celery salt
  1.0 oz = 1.0 floz - (conversion was given for 2 Tbsp)
Salt, table
  0.0141096 oz = 0.0208333 floz - (conversion was given for dash)
  10.300008 oz = 8.0 floz - (conversion was given for cup)
  0.634932 oz = 0.5 floz - (conversion was given for tbsp)
  0.211644 oz = 0.166667 floz - (conversion was given for tsp)
SUGAR PACKET
  2000.0 count = 200.0 oz - (conversion was given for a case)
Onion salt
  1.0 oz = 1.25 floz - (conversion was given for 2 1/2 Tbsp)
</similarProducts>
<targetUnit>volume</targetUnit>
<validUnits>
  quantity: count, dozen
  volume: floz, cup, tbsp, tsp, liter, gallon
</validUnits>
OUTPUT:
<reasoning>I need to a conversion that will go from quantity to volume. The product name
implies that .6 g = 1 ct, so we can use that conversion with the 16.0 oz = 12.0 floz (which
seems valid) and a known conversion 1g = 0.035274 oz to get .6 g = .6 * 0.035274 oz,
giving us the final conversion: 0.0211644 oz. We can then use the conversion 16.0 oz =
12.0 floz to get 0.0211644 oz = 0.0211644 / 16.0 * 12.0 floz = 0.015873 floz, and since we
know that the left side is equal to 1 ct, we can use that to get the final conversion:
0.015873 floz = 1 ct</reasoning>
<conversion>0.015873 floz = 1 ct</conversion>
```

### Example:

```
TABLE 1
INPUT:
<product>CREAM HEAVY WHIPPING 36% EXTENDED SHELF LIFE</product>
<amount>12/32 OZ</amount>
<similarProducts>
  No similar products found
</similarProducts>
<targetUnit>volume</targetUnit>
<validUnits>
  weight: oz, pound, gram, milligram, microgram, kilogram
  volume: floz, cup, tbsp, tsp, liter, gallon
</validUnits>
OUTPUT:
<reasoning>I need to a conversion that will go from weight to volume. Since no similar
products are there to reference, we'll have to make our best guess. Since cream is a little
more dense than water, we'll assume that 1 floz = 1.05 oz as that seems like a reasonable
conversion.</reasoning>
<conversion>1 floz = 1.05 oz</conversion>
GO!
INPUT:
<product>{product}</product>
<amount>{amount}</amount>
<similarProducts>
  {similar_products}
</similarProducts>
<targetUnit>{target_unit}</targetUnit>
<validUnits>
  {valid_units}
</validUnits>
OUTPUT:
```

Converting purchase units to recipe units can be crucial, as there are often significant differences between the two. Regardless of units, the purchased quantity of products differs from the actual usable amount for the ingredient by recipes, which is what is specified by the recipes. For instance, the weight of bananas as purchased (whole) is more than when they are peeled and chopped for a recipe. Similarly, this accounts for steps like dicing bell peppers or cracking eggs, where a percentage of food product is not usable as the ingredient and thus cannot be counted towards recipe ingredient requirements. This may be accounted for in the particular conversion used for that recipe ingredient directly. This conversion may be dependent on the product and ingredient, which determines the preparation needed to go from the product to the ingredient thus accounting for any loss. For example, a weight (ounces) of a product “whole pepper” when converted to a volume (cups) of “diced pepper” will account for an estimated loss from a portion of the “whole pepper” that is discarded.

Alternatively, it may be a separate conversion factor, for example by multiplying the converted units by a AP/EP factor. It could also be a separate conversion step that reduces the as-purchased amount units by a certain percentage after converting to the ingredient units (e.g., 1 unit actual-purchased has 0.8 units edible-portion for a particular product-ingredient-conversion). One product may have no reduction due to an edible portion when converted to be used for one ingredient but has a significant edible portion reduction when converted to be used for another ingredient (e.g., ingredient A=whole-peppers (AP/EP=1); ingredient B=diced peppers (AP/EP=0.8)). The edible portion, AP/EP, factor is useful in accurately determining how much product to order and for calculating per patient/serving recipe cost, as discussed below.

FIG. 5 illustrates a diagram of a process 500 for unit conversion, according to some embodiments of the present technology. Process 500 identifies a set of conversions for the product and automatically extracting conversion information from the description of a product.

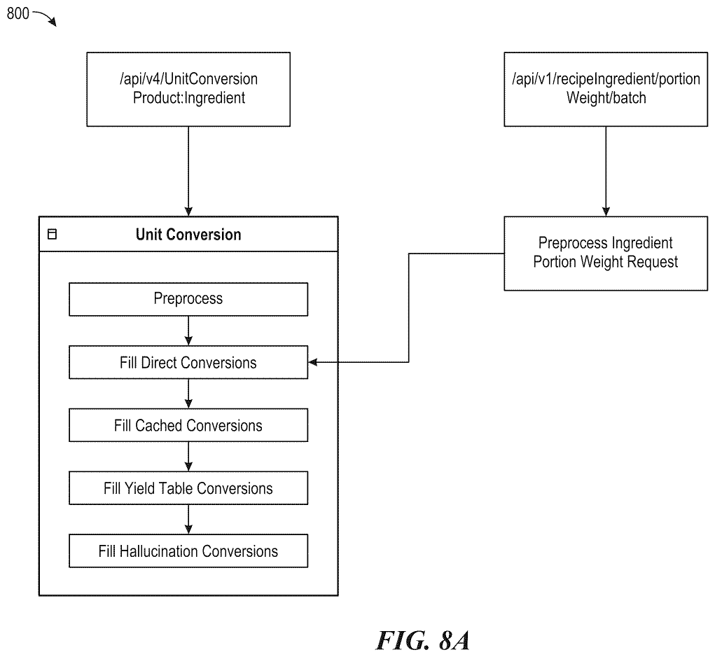

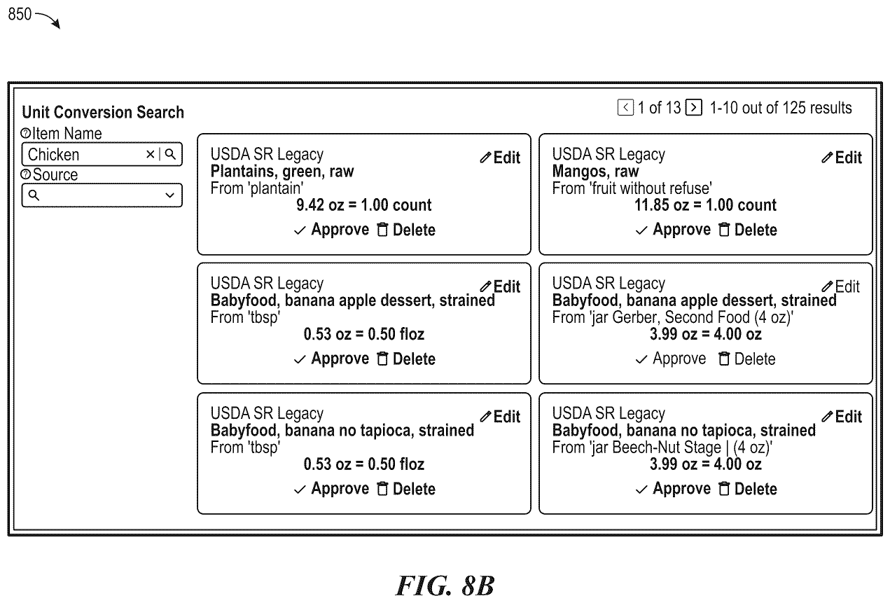

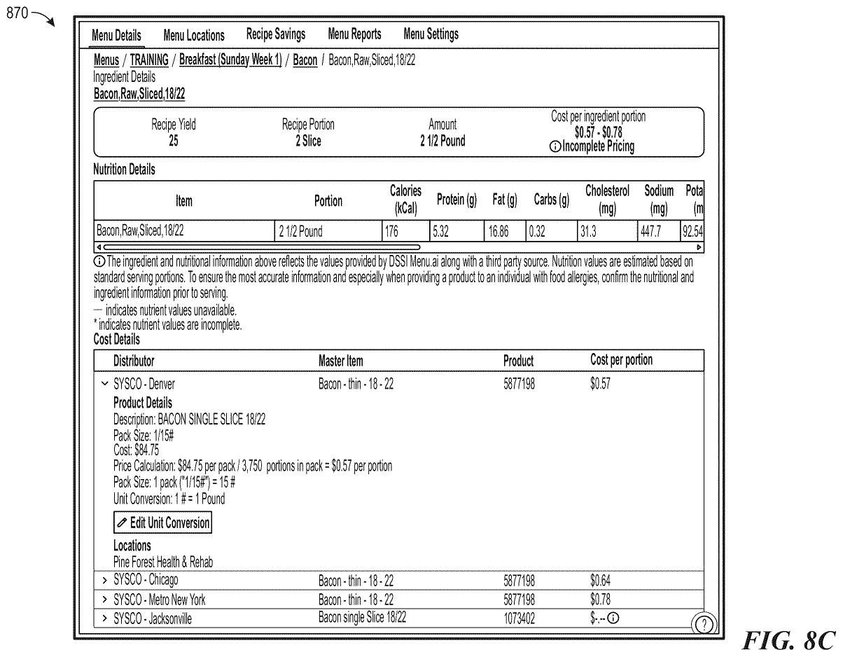

At step 502, process 500 includes receiving unit conversion input, such as product (e.g., name, description, etc.) and recipe ingredient. At step 504, process 500 includes parsing product pack size from the ingredient name and parse ingredient units from the ingredient. This identifies the units from the pack size and ingredient given a list of known units. An of example of parsing a product is: “1/50 lbs” from “Banana, Fresh 1/50 lbs.” An example of parsing an ingredient is “2 each” from “Banana, 2 each”. If the system cannot parse one or both, at step 506, the system can make a request to an LLM to attempt to parse out the pack size and units. Example: “Orange Juice, 1/52 fz” would not parsable, because “fz” is not a known unit abbreviation but could be reasoned by the LLM to mean “fluid ounces” or “fl oz” given context of the product name and ingredient. The pack size and units could then be parsed. Another example could be “Cookies, 1/12 cookies” or “Cookies, 1/12”, which would recognize that the units are “count”. LLM parsed products and ingredients can be flagged and validated internally by a user. FIG. 8B illustrates a user interface for LLM generated unit conversions.

At step 508, process 500 includes converting parsed product and ingredient to standard units. Each product and ingredient has one of three form factors: weight, volume, or quantity. And each form factor has a standard set of units: ounces for weight, cups for volume, and count for quantity. Products are first converted to standard units using a direct conversion (i.e., a conversion that stays within a single form factor). As mentioned above, this includes converting product quantities that are expressed in denominations of a particular can size (e.g., #10 can) are also converted to a volume (e.g., 12 cups), based on a list of can size to volume conversions. If canned volumes of food are inaccurate due to water content, a warning may be indicated in a UI to the end user that the conversion may be off from the product being canned food.

At step 510, process 500 includes checking for a direct conversion. The system can compare the form factor of the product and the form factor of the ingredient, and if there is a match, a direct conversion can be applied from a list. If a direct conversion is possible, at step 512, process 500 includes applying the direct conversion. Due to the product units previously being converted to standard units, the list of conversions only specifies units of the ingredient when identifying and applying the direct conversion between the product and the ingredient units.

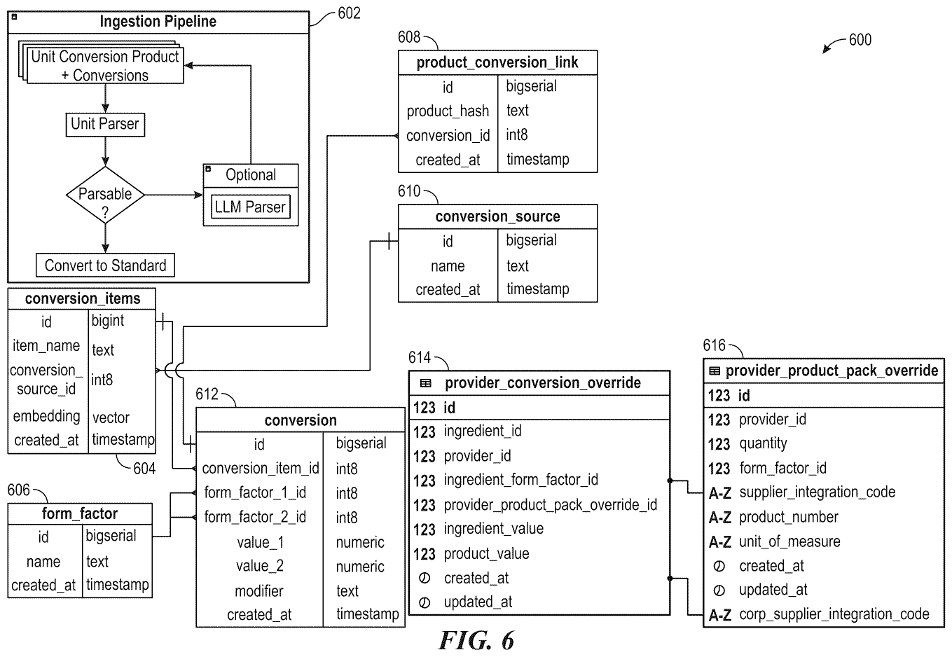

If a direct conversion is not possible/found, at step 514, process 500 includes checking for a cached conversion product reference link. If not a direct conversion, a check is done for the particular product using a product conversion link via a product hash (e.g., SHA256(Product Name+Description+Pack Size) to see if an indirect conversion for the product and ingredient form factor. This is shown in FIG. 6. This is independent of the ingredient. If a conversion exists, at step 512, process 500 includes applying the conversion.

At step 516, process 500 includes check if the ingredient form factor is quantity. If the ingredient form factor is not quality, at step 518, process 500 includes querying conversion products via embeddings. If the ingredient form factor is quantity, at step 520, process 500 includes parsing a description for conversion. The system can use a LLM to check product name and description for conversion information. Conversion information could be weight or volume form factor to quantity form factor conversion information. The LLM will output conversion values and form factors to convert the product to quantity, or indication that the product does not contain conversion information (or that it can't be determined). Some examples include:

### Example 1

Name: “Banana, Fresh, 1/50 lbs”
Description: “50 pound bag of bananas containing about 130 medium bananas”
LLM Output: 50 lbs=130 count

### Example 2

Name: “Bacon, 18/22, 1/10 lbs”
Description: “1 pound of bacon is 18-22 slices.”
LLM Output: “1 lbs=18-22 count”

The LLM output can be parsed into a conversion object, including form factors, modifier, and values. Convert form factors to their standard units and if a range is provided (e.g., “18-22 count”) then a mean value is determined.

At step 522, process 500 includes checking if a conversion is parsed from a product description. If the conversion is parsed, at step 524, process 500 includes uploading parsed conversion to a database, linking the conversion to the product, and (at step 512), applying the conversion. The source of the conversion may be indicated as being “Product Description” (or similar). This may not be considered a trusted source until it is validated manually by a user, and thus will not be surfaced using embedding search in step 518. If no conversion is parsed, at step 518, process 500 includes querying conversion products via embeddings.

At step 518, process 500 includes querying conversion products via embeddings. If either the ingredient form factor is not quantity or unable to parse a conversion from the product description, the system identifies a conversion from the database using an embedding search. The embedding search can include: 1) embed product name (e.g., “Banana, Fresh, Large”) using an embedding model; 2) calculate cosine distance with embeddings of item_name for each conversion_item; 3) filter based on conversion source (e.g., only from trusted conversion sources, such as USDA or user created (internal user)); and 4) take top five conversion items based on cosine distance.

At step 526, process 500 includes deduplicating requests. Since conversion requests are precomputed in batches, process 500 can include deduplicating input to only one product hash per target form factor, so that other requests with the same hash can use it. At step 528, process 500 includes matching via the LLM. The top conversions items, including the item_name of each, are passed in a prompt along with the product name to select the best conversion item. The appropriate conversion is selected from the conversion item based on the form factor of input product and ingredient and any modifiers. Modifiers can include product size category (e.g., small, medium, large, or a preparation step such as mashed, sliced, etc.)

At step 530, process 500 includes checking if a match is found. If a match is found, at step 524, process 500 includes uploading, to the conversion database, a reference link from the product to the conversion. The reference link may be indicated as AI selected. The conversion can then be applied (at step 512). If a match is not found, a LLM can be prompted to determine a potential conversion. At step 532, process 500 includes deduplicating requests. At step 534, process 500 includes generating (“Hallucinate”) a conversion using an LLM. The LLM is prompted to determine a potential conversion, as described FIG. 4. The term “Hallucinate” is used here, to indicate the confidence in the conversion results. This is simply used instead of a term like “generates”, which conveys more confidence in the conversion results.

FIG. 6 illustrates a unit conversion database architecture 600 and a conversion ingestion pipeline 602 are illustrated according to some embodiments of the present technology. The unit conversion database architecture 600 includes multiple interconnected database tables that store and manage conversion data for the menu optimization system. The conversion ingestion pipeline 602 receives unit conversion products and conversions from different sources and processes the conversion data for storage in the database.

The conversion ingestion pipeline 602 includes a unit parser that processes incoming conversion data to determine whether the data is parsable. If the data is parsable, the data is converted to standard units. If the data is not parsable, an optional LLM parser can be used to determine how to parse the conversion before storing the conversion in the database. The LLM parser can reason through non-standard conversion formats and extract the appropriate conversion values and form factors.

The unit conversion database architecture 600 includes a conversion items table 604 that stores conversion item records and a form factor table 606 that stores form factor definitions. Form factors represent the measurement categories such as weight, volume, or quantity. The form factor table 606 provides a reference for the form factor identifiers used in the conversion table 612.

With continued reference to FIG. 6, the unit conversion database architecture 600 includes a product conversion link table 608 that links products to conversions. The product conversion link table 608 enables the system to cache and retrieve previously determined conversions for specific products, reducing the need for repeated conversion calculations. The unit conversion database architecture 600 includes a conversion source table 610 that stores information about the sources of conversion data. Each conversion source can have many associated conversion items. Examples of conversion sources include USDA conversion tables, user-created conversions from internal users, and conversions parsed from product descriptions.

The unit conversion database architecture 600 includes a conversion table 612 that stores the actual conversion records. The conversions database is made up of multiple conversion items each with multiple conversions, where each conversion can be linked to one or more products with a corresponding product conversion link. The unit conversion database architecture 600 includes a provider conversion override table 614 that allows providers to override conversion values. The provider conversion override table 614 enables customization of conversion values for different providers based on their specific product configurations or preparation methods. The unit conversion database architecture 600 includes a provider product pack override table 616 that stores provider-specific product pack information. The provider product pack override table 616 enables customization of product pack configurations for different providers, accommodating variations in how products are packaged or measured across different suppliers and distribution centers.

Accurate unit conversions can be critical in determining the actual cost per serving of a recipe. This is done by calculating the per-serving cost of each ingredient from the corresponding product amount and price, in a recipe and combining the price of the ingredients to get the total cost of the recipe per serving. The system can pull the cost of the corresponding product, which is dependent on the specific customer. The cost of the corresponding product can be used to calculate cost per patient day (PPD) and be used for recipe cost comparison, and optimization with recommendation prioritization.

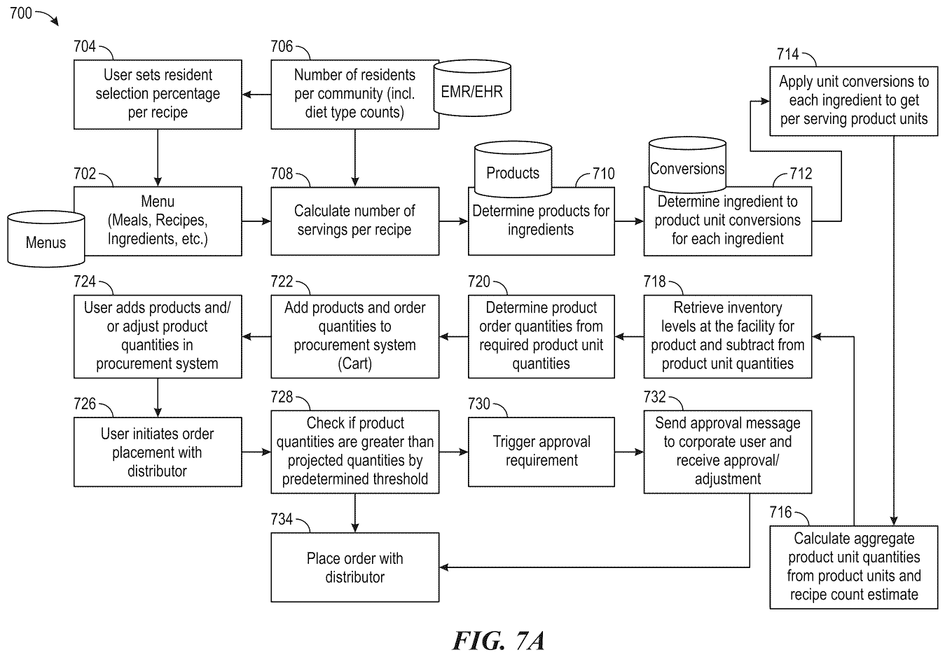

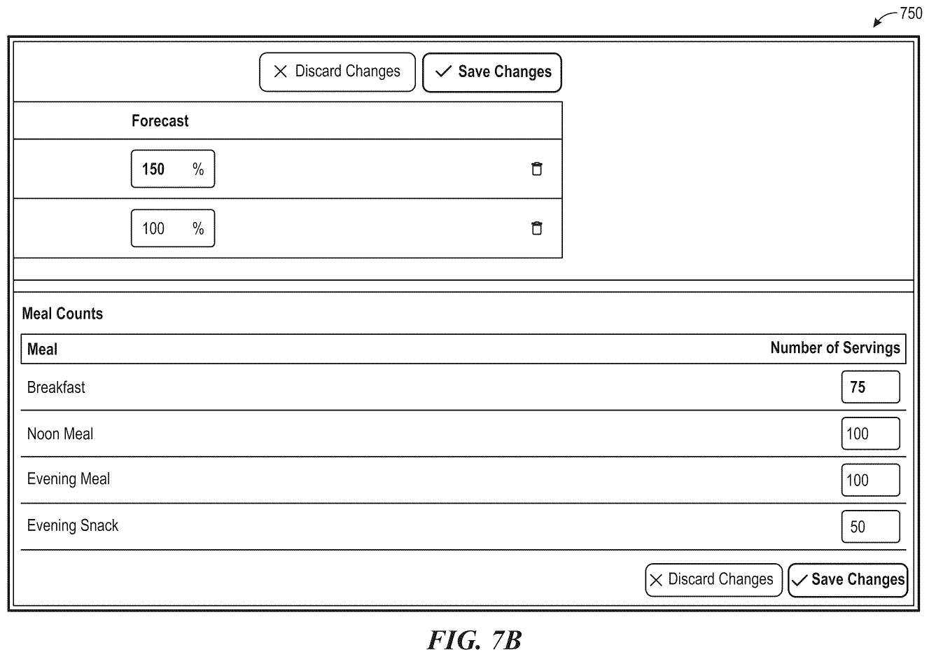

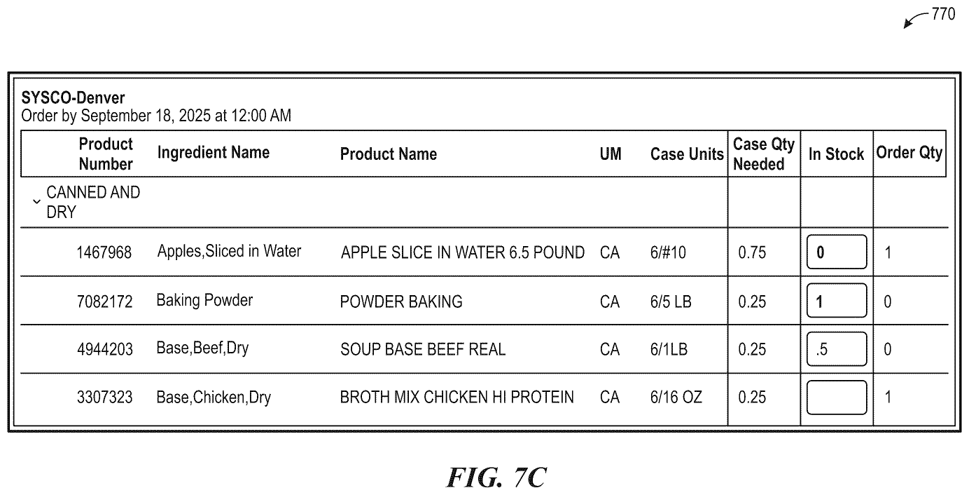

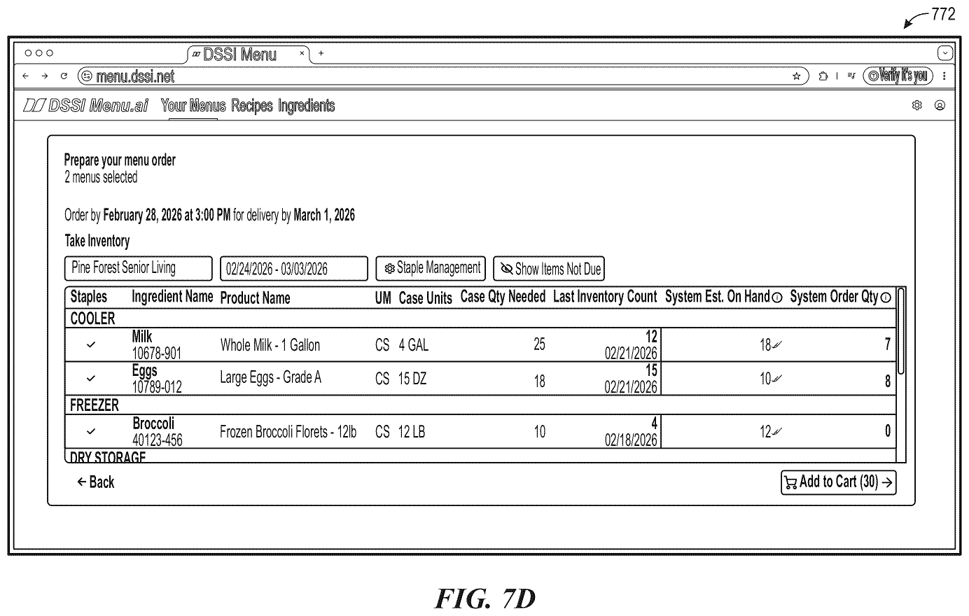

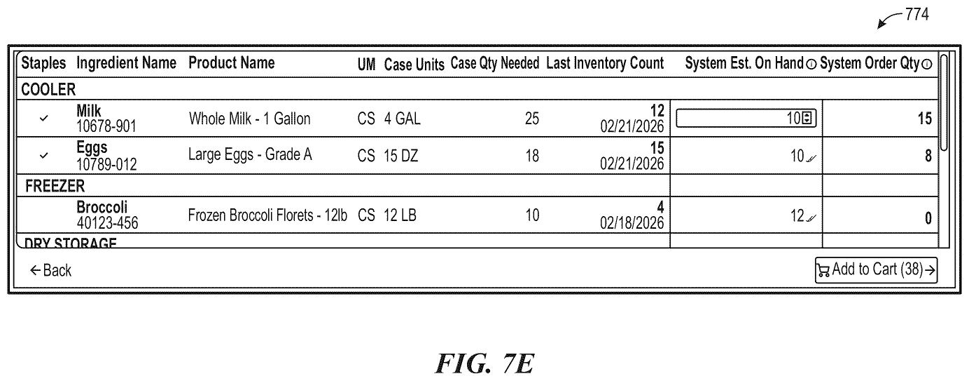

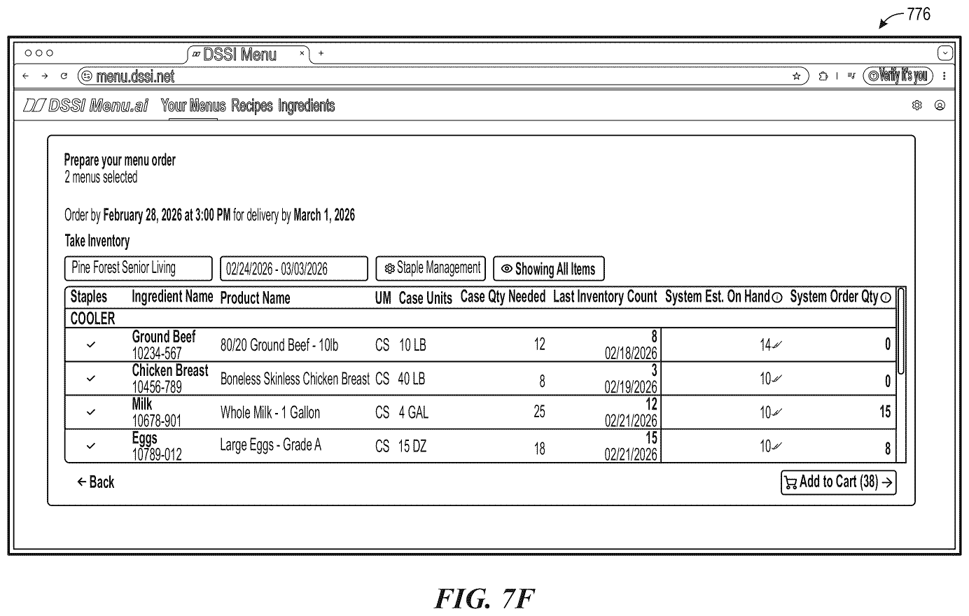

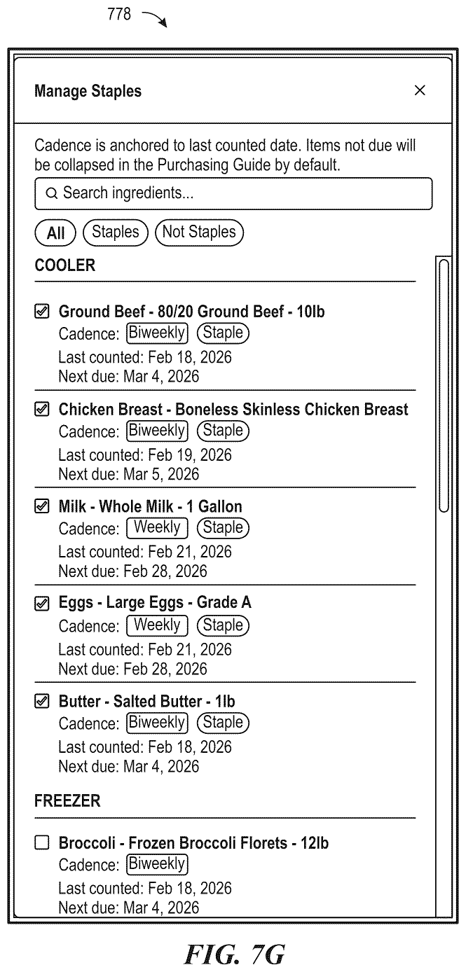

FIG. 7A illustrates a diagram of a process 700 for automating product ordering based on menus, according to some embodiments of the present technology. The number of servings of each ingredient for a recipe is used to determine the quantity of corresponding product to order. This is crucial when determining the quantity needed to prepare enough servings of a recipe for the facility's residents. Process 500 can be used to calculate the required product quantities.

At step 702, process 700 includes retrieving menu recipes. For the specified timeframe, the system can retrieve meals, recipes, and corresponding percentages. This includes percentage of residents that are predicted to “select” (aka eat) each recipe. This may be filtered based on various factors. At step 704, process 700 can include receiving a user set resident selection percentage per recipe. At step 706, process 700 includes determining or receiving the number of residents per community.

At step 708, process 700 includes calculating the number of servings needed per recipe. The system can assess the population that will consume a serving of the recipe, considering the community census, and percent of community that will select each recipe (e.g., as set by a user, such as a chef or dietitian) an/or determined from diet type spread (e.g., retrieved from an external system, such as an EMR/EHR). The initial resident percentages may be determined and set by a corporate user for multiple facilities but may be adjusted or overridden by each facility based on preferences. These percentages determine the number of servings/portions of each recipe needed, and thus the number of servings/portions of each ingredient needed. Community census (e.g., from EMR/HER, counts of residents by diet type, etc.) can include a total number of residents by community, building, etc. Percent of residents for each recipe of each meal can include 1) some recipes are specific to diet types, and the count/percentage of residents can be estimated from diet types; 2) initial recipe percentages are set by a corporate user; and 3) adjusted/overridden for specific community by a community user (chef, dietitian, etc.).

At step 710, process 700 includes determining products for ingredients. Products are determined for the ingredients using the product to ingredient linking model, as described above. Note some ingredients for different recipes will be linked to the same (or similar) product and thus can be aggregated when calculating the product quantities. At step 712, process 700 includes determining ingredient to product unit conversions for each ingredient.

At step 714, process 700 includes applying unit conversions to each ingredient to get per serving product units. Accounted for by the conversion, the portion of food needed for each serving is based on the edible portion of the product. This accounts for the percentage of the product that is usable as an ingredient. This effectively means accounting for loss when determining how much to order. This is part of the conversion process from product to ingredient units, and vice versa. In some cases, when converting the quantity of ingredient units to the required quantity of each product to purchase, the quantity is divided by the edible portion percentage (AP/EP factor).

At step 716, process 700 includes calculating aggregate product unit quantities from product units and recipe count estimates. For each product the converted units are multiplied by the number of required servings, as determined above, to arrive at the quantity of as-purchased units of the product.

At step 718, process 700 includes retrieving inventory levels at the facility for the product and subtract the inventory levels from product unit quantities. The system can compare the required purchased quantity of product to inventory levels of the product at the community from an inventory management system and subtract current inventory levels for each product to reduce order quantities. This can help to minimize over-ordering of products and waste. Current inventory levels may be manually entered by a user for the specific products identified or pulled from an inventory system. In some embodiments, the system may determine whether manual inventory verification is required before proceeding with order quantity calculations. This determination can be based on factors such as the recency of the last inventory count for a product, the typical usage patterns of the product, recent receiving activity at the facility, or whether the product is categorized as higher risk due to cost, perishability, or regulatory requirements. For example, a product that was counted within the past week and has predictable usage patterns may not require verification, while a product with an outdated count or irregular consumption may prompt the system to request manual confirmation before the user can proceed. The system may display indicators in the user interface identifying products that require inventory verification and may prevent order submission until the verification is completed.

Over time, the system may refine inventory confidence assessments based on expected usage derived from planned menus, recent receiving records, and known waste or spoilage patterns, rather than relying solely on static inventory counts. The system can track the difference between expected inventory levels and actual counted levels to identify products with higher variance, which may warrant more frequent verification. Products with consistent alignment between expected and actual inventory may be flagged as lower risk, allowing users to proceed without manual verification steps. This approach enables the system to adapt verification requirements dynamically based on historical accuracy and operational patterns at each facility, reducing unnecessary verification overhead while maintaining accuracy for products where inventory data is less reliable.

In some embodiments, products may be assigned product-level tags or attributes that influence how the system manages inventory verification requirements for those products. For example, a product may be tagged as a staple item, indicating that it is regularly used and has predictable consumption patterns, or as a high-risk item, indicating that it warrants more careful inventory management due to factors such as cost, perishability, regulatory sensitivity, or consumption variability. These product-level tags do not change the underlying calculations for order quantities but instead inform the system's expectations about when manual verification is appropriate versus when it is reasonable to proceed based on estimated inventory levels.

For products tagged as high-risk, the system may apply stricter inventory confidence thresholds, such that the confidence in the last inventory count decays more quickly over time and manual verification is prompted sooner than for standard products. The system may also generate stricter recommendations prior to ordering for high-risk products, such as requiring verification even when the last count is relatively recent. In contrast, products tagged as staples with consistent usage patterns may be allowed to proceed with longer intervals between manual counts, as the system can more reliably estimate current inventory based on planned menu usage and receiving records.

The product-level tags may also be used in audit scenarios, where a user may choose to perform an inventory count limited to products tagged as high-risk rather than counting all products at the facility. This targeted approach allows facilities to focus verification efforts on products where accuracy is most important while reducing the burden of counting lower-risk items with predictable inventory levels. The user interface may provide filtering options that allow users to view and manage products by tag category, enabling efficient workflows for both routine inventory management and periodic audits.

At step 720, process 700 includes determining product order quantities from required product unit quantities. The product unit quantities can be used to determine the quantity of packs/cases of the product for ordering. At step 722, process 700 includes adding products and order quantities to a procurement system (e.g., a cart). This may be done directly within the system through a fully integrated procurement system, or by passing a message via an API to a separate procurement system.

At step 724, process 700 includes the user adding products and/or adjusting product quantities in the procurement system. At step 726, process 700 includes the user initiating order placement with the distributor. The user may place the order, which is sent to the distributor for fulfillment. Before being sent there may be checks of products and product quantities against the calculated order quantities and trigger approval by a corporate user. For example, at step 728, process 700 includes checking if product quantities are greater than projected quantities by a predetermined threshold. In addition to cost or quantity-based approval checks, the system may also determine whether inventory verification is required before the order is submitted. This verification step is distinct from order approval and focuses on whether the available inventory context is sufficiently reliable to proceed with the calculated order quantities. The system may evaluate factors such as the age of the last inventory count for each product, the historical variance between expected and actual inventory levels, and whether any products in the order are flagged as requiring periodic verification due to cost, perishability, or consumption variability. If the system determines that inventory data for one or more products is stale or unreliable, the user interface may prompt the user to confirm current inventory levels for those specific items before allowing the order to be submitted. This approach ensures that order quantities are based on accurate inventory information while avoiding unnecessary verification steps for products where inventory data is consistently reliable.

At step 730, process 700 includes determining a trigger approval requirement based on comparison of order to various factors. For example, order quantity is larger than expected quantities based on corporate user set recipe selection percentages. At step 732, process 700 includes sending an approval message/notification along with relevant information (e.g., products, order quantities, projected order quantities). At step 734, process 700 includes placing an order with a distributor. A new order module may be displayed to a food procurement employee, to place an order for food. It can be triggered based on a scheduled time ahead of a known, planned order delivery date. The order is intended to generally include food items needed for the menu for a timeframe up until the next food delivery date.

The system can determine the number of servings of each recipe required based on census data (e.g., facility population). This can account for the count of various diet types of residents (i.e., diet spread) as not all recipes can and will be consumed by every resident. Some special dietary recipes will be required by a smaller number of residents and thus far fewer servings will need to be made, and thus corresponding products purchased

In some embodiments, there may be multiple menus for a single community. For example, there may be a different menu for each diet type, where each menu may specify recipes for that diet type. The products and product quantities can be determined for each menu and aggregated for product ordering

The percentage of residents that are anticipated to select (i.e., want) each recipe, may be edited and adjusted by a community user (e.g., chef, dietitian) within the menu system before adding products and product quantities to the procurement system. Similarly, the number of servings for each meal can be selected by user, or could similarly be as a percentage of the total number of residents. Alternatively or additionally, entire recipes may be changed. In addition, or alternatively, once the products and corresponding quantities are added to the procurement cart, the products and quantities may also be adjusted. This can be done to reflect known preferences of residents with the particular community. This adjustment can be tracked and provided as feedback to the corporate user preparing the corporate-level menu for future menu planning (e.g., a recommendation to swap a particular recipe with another recipe based on community adjustments feedback).

In some cases, the community user may try ordering more than projected based on the corporate user set resident selection percentages for a recipe/meal for a community using the above calculations. This can trigger an approval process by the corporate user, sending a message requiring that the order, or portion of an order, be approved. The corporate user may approve, or decline the order, including adjusting the product, product quantity, then submit the order. A message can be sent back to the community user from the corporate user indicating why the order or portion of order was declined.

Similarly, changes to the menu, such as changing out a recipe, may trigger an approval request to a corporate user. The approval may be sent as a message including the change, which products are being swapped, as well as relevant context from the menu, which the corporate user may approve or reject via input elements. A message may be sent back to the community user with an explanation for the approval/rejection decision. The approval request can be configured to only be triggered if the recipe change increases the cost by a threshold raw amount or percentage, and/or the recipe being swapped to is not nutritionally equivalent (as discussed below).

In some embodiments, the system may support subrecipes, where a recipe can be included as an ingredient within another recipe. For example, a salad recipe may include a homemade dressing as one of its ingredients, where the dressing itself is defined as a separate recipe with its own ingredients, preparation steps, and yield information. When calculating product quantities for a parent recipe that includes a subrecipe, the system can recursively traverse the subrecipe to identify all underlying ingredients and their required quantities. The subrecipe's yield and portion information can be used to determine how much of the subrecipe is needed for each serving of the parent recipe, and the ingredient quantities from the subrecipe can be scaled accordingly. This approach allows for reuse of common preparations across multiple recipes, such as sauces, stocks, or spice blends, while maintaining accurate cost calculations and nutritional analysis at both the subrecipe and parent recipe levels. The unit conversion and product linking processes described above can be applied to subrecipe ingredients in the same manner as direct recipe ingredients, with the aggregated product quantities reflecting the total requirements across all recipes and subrecipes in a menu.

Referring to FIG. 7B, a meal counts interface 750 is illustrated according to some embodiments of the present technology. The meal counts interface 750 provides user controls for setting forecast percentages and number of servings per meal, which are used in calculating the quantity of products needed for ordering. The meal counts interface 750 includes a forecast section that allows users to set a forecast percentage for each meal, such as 150% for Breakfast and 100% for Noon Meal, where the percentage represents the anticipated proportion of residents expected to select that meal. The meal counts interface 750 also includes a meal counts section that displays and allows editing of the number of servings for each meal period, such as 75 servings for Breakfast, 100 servings for Noon Meal, 100 servings for Evening Meal, and 50 servings for Evening Snack. The forecast percentages and number of servings can be set at the meal level and may also be configured at the individual recipe level within each meal. These values may be manually entered by a user such as a chef or dietitian, or may be estimated and pre-populated based on diet type counts derived from resident or patient profile data. In some aspects, the diet type counts may be retrieved from an electronic medical record (EMR), electronic health record (EHR), or resident information database that maintains information about each resident's dietary requirements and restrictions. The system uses the configured forecast percentages and serving counts in combination with recipe ingredient information and unit conversions to calculate the aggregate product quantities needed for procurement.

Referring to FIG. 7C, a purchasing guide interface 770 is illustrated according to some embodiments of the present technology. The purchasing guide interface 770 displays a purchasing guide generated from menu ingredients for a specified order period. The purchasing guide interface 770 shows order information including the distributor (e.g., SYSCO-Denver) and an order deadline. The purchasing guide interface 770 presents a table organized by product category (e.g., “CANNED AND DRY”) that lists products derived from the menu ingredients, including columns for product number, ingredient name, product name, unit of measure, case units, case quantity needed, in stock quantity, and order quantity. The case quantity needed column reflects the calculated quantity of each product required based on the menu recipes, serving counts, and unit conversions as described above. The purchasing guide interface 770 provides the ability for a user to enter the quantity of each product that is currently in stock at the facility through editable input fields in the in stock column.

The in stock quantities may be manually entered by a user or may be pulled from an inventory management system that tracks current inventory levels at the facility. The system adjusts the menu-based estimate by the in stock quantity entered for each product to determine the order quantity. For example, if the case quantity needed is 0.25 and the in stock quantity is 1, the order quantity may be set to 0 since sufficient inventory exists. The purchasing guide interface 770 allows users to review and modify the calculated order quantities before adding the products to a cart of a procurement system. Once the user has reviewed and adjusted the quantities as needed, the products and order quantities can be added to the procurement system cart, and the user can complete the order as normal through the procurement system to place the order with the distributor for fulfillment.

Referring to FIG. 7D, a user interface 772 for preparing a menu order is illustrated according to some embodiments of the present technology. The user interface 772 displays order preparation information including the number of menus selected, order deadline, and delivery date for a specified location. The system estimated on hand column reflects an inventory estimate calculated by the system based on recent orders placed for the product, typical historical usage patterns derived from past consumption data, and expected waste or spoilage factors associated with the product type. The system order quantity column reflects a calculated order quantity based on menu plans for the upcoming period, including menu recipes organized by meals, forecast percentages indicating anticipated resident selections, and meal counts specifying the number of servings per meal, with the calculated quantity then adjusted by the current inventory as reflected in the system estimated on hand value.

Referring to FIG. 7E, a user interface 774 for a purchasing guide is illustrated according to some embodiments of the present technology. The user interface 774 displays a purchasing guide with columns including system estimated on hand quantities that may be manually adjusted by a user based on manual review. The system may flag and alert the user to conduct a manual review of specific products based on a confidence score that falls below a threshold value. The confidence score may be determined based on factors including item classification and time since the last inventory count for that product. For example, products classified as high-value, perishable, or having variable consumption patterns may have confidence scores that decay more rapidly over time, prompting earlier manual verification. Similarly, products for which the last inventory count occurred beyond a configured time threshold may be flagged for review regardless of item classification. When the system determines that the confidence score for a product falls below the threshold, the user interface 774 may display a visual indicator adjacent to the system estimated on hand value, alerting the user that manual verification is recommended before proceeding with the order. The user may then enter an updated quantity in the system estimated on hand field based on a physical count or other verification, and the system order quantity is recalculated accordingly.

Referring to FIG. 7F, a user interface 776 for preparing a menu order and managing inventory is illustrated according to some embodiments of the present technology. The user interface 776 provides functionality for users to selectively display all items for a selected location and timeframe, including items for which the system calculates a zero order quantity. The user can view the complete list of products associated with the menu for the specified period, rather than only those products that require ordering. This comprehensive view allows users to review products such as “Ground Beef” and “Chicken Breast” that have sufficient inventory on hand, as indicated by a system order quantity of 0, alongside products like “Milk” and “Eggs” that require additional ordering. Displaying items with zero order quantities enables users to verify that the system's inventory estimates are accurate, confirm that no additional product is needed for items with adequate stock, and identify opportunities to adjust inventory levels or meal plans based on the full product picture. The user interface 776 presents each product with its ingredient name, product name, unit of measure, case units, case quantity needed based on menu calculations, last inventory count date, system estimated on hand quantity, and system order quantity, allowing users to make informed decisions about whether to accept the calculated order quantities or make manual adjustments.

Referring to FIG. 7G, a user interface 778 for managing staples is illustrated according to some embodiments of the present technology. The user interface 778 provides functionality for managing which items at a location are considered staples and configuring the cadence by which inventory quantities for those items should be reviewed and updated. Staples are items that are not desirable to count every time inventory is performed because they are typically always on hand at the facility, such as salt, spices, or large bulk items like a 50 pound bag of flour. The user interface 778 displays a list of ingredients organized by storage location, such as cooler, freezer, and dry storage, with each item showing its ingredient name, product name, and associated staple designation. For items designated as staples, the user interface 778 displays a configured cadence indicating how frequently the item should be counted, such as weekly or biweekly, along with the date of the last inventory count and the next due date for counting based on the configured cadence.

Ordering frequently may slow down at the point of order placement because users lack sufficient confidence in available inventory information. Even when required quantities are known, users often perform manual inventory verification, such as physical counts or secondary checks, before submitting an order. This verification step introduces delay, inconsistency, and operational overhead, particularly for high-risk or variable items. inventory systems may provide data but often do not determine whether manual verification is required at the ordering moment.

The system may address this challenge by conditionally bypassing manual inventory verification during order placement based on item-level confidence thresholds derived from multiple inventory signals. At the ordering moment, the system may generate a usage-aware inventory estimate by aggregating menu-planning-based required quantities, item classification, recent inventory counts, historical usage, receiving events, and expected waste. The system may evaluate whether the available information satisfies a confidence threshold associated with the item type. Based on this evaluation, the system may either permit the user to proceed directly to ordering or require manual inventory verification before order submission.

The system may determine a required inventory quantity for an item based on planned consumption derived from menu plans, including meals, recipes, forecasts, and meal counts. The system may identify an item classification, such as high-risk, staple, or seasonal, that corresponds to a confidence requirement for ordering decisions. The item classification may be assigned at the order guide management level through tagging, with system-suggested classifications that users can adjust as needed. The system may retrieve a most recent verified inventory state and a time elapsed since the verification.

The system may generate an estimated remaining inventory quantity or range by adjusting the last verified inventory state using historical consumption data, recent receiving events, and expected waste. The system may determine whether the estimated remaining inventory satisfies a confidence threshold associated with the item classification, including consideration of time-based confidence decay. Based on the evaluation, the system may either permit order placement without manual inventory verification or require manual inventory verification prior to order placement. This approach may enable users to proceed efficiently with ordering for items where inventory confidence is high while ensuring appropriate verification for items where inventory data is less reliable.

FIG. 8A illustrates a diagram for an ingredient unit to nutrients conversion process, according to some embodiments of the present technology. The process for identifying the proper conversion for ingredients units to nutrients or USDA weight/volume units (i.e., ounces or cups) is generally very similar to the process used for product to ingredient conversions. However, they can differ in intent, thus changing the preprocessing and post processing. Instead of going from product units to ingredient units, the system can match ingredients up to known nutritional records (e.g. the nutritional facts for strawberries) as provided by some authoritative source (e.g. the USDA) in a given quantity (e.g. 100 g).

More specifically, the quantity of each nutritional can be calculated for a single portion of the ingredient for the recipe. These nutritionals include macronutrients, such as calories, and grams of carbs, protein, fats, or sugar; and/or micronutrients (as described further below). Alternatively, the nutritional information can be USDA equivalent-weight/volume of the appropriate USDA my plate category: protein, vegetable, fruit, grain, dairy (as described further below). The nutritional quantities for each ingredient can then be aggregated for the recipe and can be used to determine the nutritional similarity between recipes for recipe recommendations. These conversions are useful for analyzing recipes, meals, and menus for nutritional spread, quality and regulatory compliance (e.g., creating reports to show regulatory compliance).

FIG. 8A shows the standard product to ingredient unit conversion process 800 and the nutritional unit conversion process 802. This highlights the high-level similarities and differences. The pre-processing differs from the product-ingredient conversion, instead of the input being a product with units that need to be converted to ingredient units. The input is an ingredient and a target conversion. The ingredient includes a name and units, and the target conversion is the quantity tied to a nutritional record stored by the system (e.g., 100 g of strawberries has x calories, y sugar, etc.) This differs from standard product to ingredient conversions, as there is no need to parse pack size or convert cans.

The core steps are generally similar to the product-ingredient unit conversions, as described above, the appropriate conversion falls into one of the following tiers of conversions: direct conversion, user-provided conversion, indirect (yield table) conversion, and non-standard (LLM “hallucination”) conversion. Direct conversion can include checking whether it is a direct conversion in a list of direct conversions, based product units and ingredient units. Indirect conversion can include checking for a density conversion match in the yield table using an embedding search, requiring a minimum of a 90% match score threshold to be met. The top match is selected by some selector (e.g. top similarity score, an LLM reranker, etc.). Non-standard conversion can include passing units of ingredient and corresponding USDA weight units to a LLM, along with the closest conversions identified from the yield table based on embedding search results, to reason through the conversion, and provide an estimate for the conversion.

The post processing also differs from the product-ingredient unit conversion, as it always wants the target to be in gram units. The core unit conversion process converts to either ounces or grams, it can then be converted to grams. For any ingredient, it will be quantified in gram units for a single serving or portion.

To determine the quantity of each nutritional for a particular ingredient, the ingredient is first related to a USDA food item in a database relating each ingredient to its quantity of each nutrient for a 100 g unit of that food item. A match is found by doing an embedding search using the ‘name’ of the ingredient as the query embedding, and ‘name’ for the USDA food item using cosine similarity to calculate a similarity score between the embeddings. If a match is found based on a threshold value (e.g., >0.8 score), then parsed nutritional quantities can be used to calculate the quantity of each nutrient for the ingredient, as described in the next step.

Using units of the ingredient is converted to 100 g units, and if the system identifies a USDA food item in the database and relates the units back to the amount of each nutritional for the ingredient. For example, grams of protein per 100 g portion of an ingredient (e.g., more specifically, 25 g of protein for a 100 g unit of chicken breast). The nutritionals can be summed up for all ingredients in the recipe for a single portion. The total nutritionals of the recipe can be used for determining nutritionally similar recipes, as described below.

Nutritional information for ingredients is pulled from general USDA information for food ingredients. Additionally, information can be determined from the specific linked product, if available. Nutritional information can include: 1) specific quantities of nutrients, such as macro and micronutrients, or USDA my plate categories (e.g., conversion process above is used for determining ingredient nutritional quantities and then recipe nutritional quantities); and 2) tags such as meal pattern group, specific dietary compliance, etc. (e.g., some tags can also be determined from product name or description, ingredient name, recipe name, etc.).

Although dietitians largely use food group categories for determining nutritional quality of recipes, meals, etc. Macronutrient and micronutrient may also be determined for a single portion of recipe based on ingredient conversions and aggregation. Macronutrients (macros) are nutrients needed in larger amounts to provide energy and maintain bodily functions. For example, this includes (1) carbohydrates, such as breads, pastas, fruits, and vegetables; (2) proteins, found in meat, fish, eggs, dairy, legumes; (3) lipids, i.e., fats, found in oils, nuts, seeds, and fatty fish; and (4) sugar. It could also include (5) water and (6) fiber. Micronutrients (micros) are nutrients needed in smaller amounts but are still vital to health. They include (1) the various vitamins, and (2) minerals such as calcium, potassium, iron, and zinc.

The USDA MyPlate divides foods into five categories: fruits, vegetables, grains, protein foods, and dairy. Each category has specific recommendations for daily intake, represented in cup equivalents for fruits, vegetables, and dairy, and ounce equivalents for grains and protein foods. For example, one cup of raw or cooked vegetables or vegetable juice counts as one cup from the vegetable group, while one slice of bread or one ounce of ready-to-eat cereal counts as one ounce from the grains group. This system helps individuals balance their diet by ensuring they consume a variety of nutrients from different food groups.

There may also be a separate process for classifying recipes into food groups for dietitians. Classifying ingredients and/or recipes into one or more broad food groups (USDA My Plate Categories) is useful to ensure a balanced and nutritious diet. The ingredients can then be converted into their USDA equivalent weight/volume units (using the conversion identified above). This provides dietitians with broad nutritional impact (e.g., impact on “cups of fruit”, “cups of vegetables”, “ounce-equivalent of protein”, “ounce-equivalent of grains”, “teaspoons of oil”, etc.) to comply with regulation, which specifies amounts of each food category required each meal/day. Accordingly, the quantity of each particular food group can be determined for each recipe, meal, day, etc. using the conversions. This information can be displayed in a user interface along with other nutritional information to provide an overview of this information for the specified granularity.

The nutritional information of recipe ingredients can be used when determining substitute or alternate recipes. The nutritional information of an ingredient can be determined from the linked product (if available) or from generic USDA information for the particular ingredient. This is important when determining alternate recipes for residents with diet types, dietary restrictions, and preferences that differ from the standard diet. In many cases, specific ingredients or quantities of ingredients may be required for a diet type or conversely make a recipe unsuitable for a diet type.

There is also for some recipes and products a difference between the recipe units and the final prepared-portion units. For example, uncooked and cooked weights/volume, such as, units of uncooked rice vs cooked rice, units of uncooked meat vs cooked meat, etc. There may be a separate conversion step that is used when determining nutritional amounts but is not used for determining product ordering or recipe costing, which instead needs to account for as-purchased units and the raw ingredients units (accounting for edible portion, AP/EP factor).

Referring to FIG. 8B, a unit conversion search interface 850 is illustrated according to some embodiments of the present technology. The unit conversion search interface 850 provides functionality for managing and validating unit conversions within the menu system, including conversions that may have been generated by a language model. The unit conversion search interface 850 includes a search panel with filtering options for item name and source, allowing users to locate specific conversions within the database. Search results are displayed with pagination controls to navigate through large sets of conversion entries. Each conversion entry displays the conversion item name, source information, and the conversion values between different units of measure. The unit conversion search interface 850 provides user controls for human validation of conversions, including options to approve or delete each conversion entry, as well as an edit button that allows users to manually modify conversion values. In some aspects, conversions generated by a language model may be presented through this interface for review by a dietitian or other qualified user before being incorporated into the system. Approved conversions, deleted conversions, and manually edited conversions may be stored and used as training data for fine-tuning the language model, thereby improving the accuracy of future conversion generation. This human-in-the-loop validation approach allows the system to leverage automated conversion generation while maintaining quality control through expert review.

Referring to FIG. 8C, an override user interface 870 is illustrated according to some embodiments of the present technology. The manual override user interface 870 provides functionality for viewing ingredient details, product linking information, and unit conversion data, as well as for manually adding or overriding unit conversions when automated conversions are unavailable or inaccurate. The manual override user interface 870 displays recipe information including recipe yield, recipe portion, and ingredient amount, along with nutrition details showing values such as calories, protein, fat, carbohydrates, cholesterol, sodium, and potassium for the ingredient. The manual override user interface 870 presents cost details including distributor information, product description, pack size, cost, and price calculation showing how the cost per portion is derived from the pack cost and portions per pack. The manual override user interface 870 includes an edit unit conversion option that allows users to create unit conversion overrides by specifying how much product is in a pack and how much total product the recipe is calling for, with fields for entering amounts and selecting units such as count, weight in ounces, or volume in fluid ounces. In some aspects, the manual override user interface 870 may include an option to apply the override at all distribution centers or to specific distribution centers. The manual override user interface 870 also displays location-specific information showing different master items, product numbers, and costs per portion across multiple facilities and distributors. This manual override capability forms part of the human validation process, allowing dietitians, chefs, or other qualified users to correct or supplement automated unit conversions when the system's automated conversion tiers do not produce accurate results for particular product-ingredient combinations.

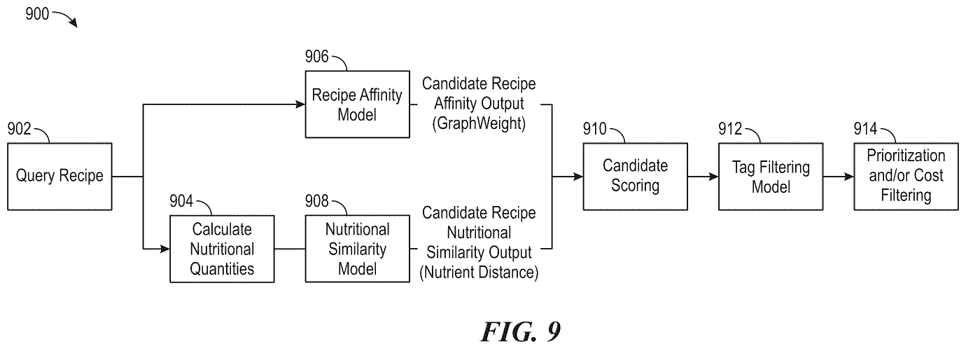

FIG. 9 illustrates a diagram of a recommendation model 900, according to some embodiments of the present technology. The model 900 determines similar recipes that can be used as optimized substitutes for a particular recipe in a meal of a menu. The recommendation model 900 includes models that are used for determining appropriate potential recipe recommendations using three components: nutritional similarity model 908; recipe affinity model 906; and recipe tag filtering model 912. The calculation for combining each of these into a single candidate score is described further below. The recommendation model 900 can query a recipe (902), calculate nutritional qualities (904), and use the nutritional similarity model 908 to generate candidate recipe nutritional similarity output. The recipe affinity model 906 can use the recipe query to generate candidate recipe affinity output. The recommendation model 900 uses the candidate recipe affinity and nutritional similarity output to determine candidate scoring (910). The tag filtering model 912 filters and tags the candidate scoring output and the filtered/tag recipe is prioritized and/or cost filtered (914).

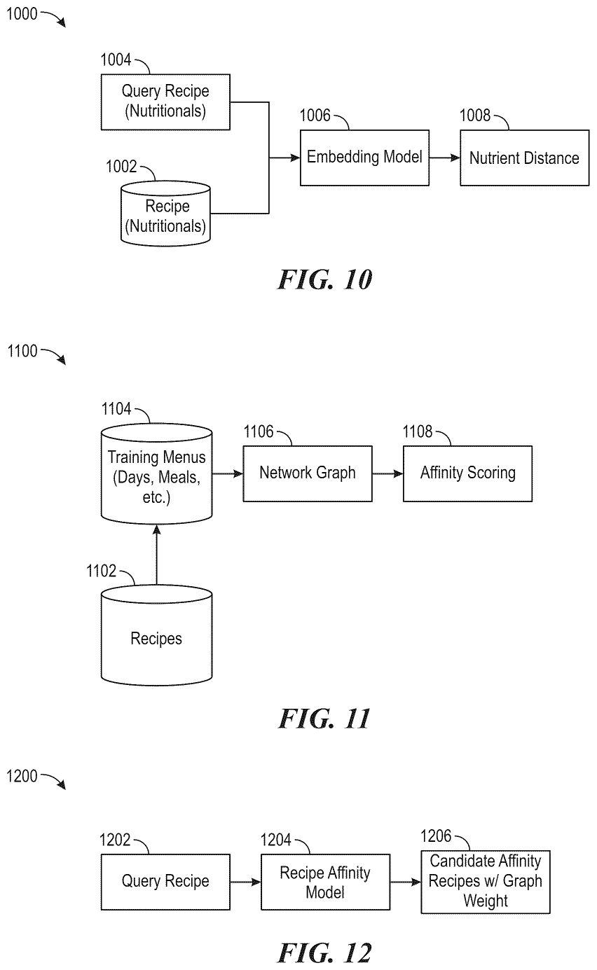

FIG. 10 illustrates a diagram of a process 1000 for generating candidate recipes, according to some embodiments of the present technology. Process 1000 can determine a similarity score between nutritional information of the recipe. This model determines candidate recipes that are nutritionally equivalent or similar enough to a query recipe.

At step 1006, process 1000 includes generating embeddings for nutritional information of the query recipe (1004) and candidate recipe (1002) alternatives. Embeddings can be generated using various methods (e.g., neural network encoder; word embedding model; contextual embedding models, etc.). Embeddings can be based on quantities of core nutrients (e.g., calories, and standard units of core nutrients: carbs, protein, fat, and sugar). It could also include quantities of micronutrients, or based on dietitian food classification group, such as USDA standard units of USDA My Plate categories.

At step 1008, process 1000 includes determining the nutrient distance (similarity score). Process 1000 can include calculating normalized distance as a similarity score between query embedding and candidate embeddings (e.g., using Euclidian distance, cosine similarity, etc.). Process 1000 can include applying a threshold value to filter out candidate recipes based on the calculated nutrient distance. This could also be a top percentage of candidates, or number of candidates, with a minimum number of candidates.

FIG. 11 illustrates a diagram of a process 1100 for constructing a network graph, according to some embodiments of the present technology. The likelihood of recipes (food items) appearing together in a cycle menu (e.g., in the same meal). This model determines meal compatible alternatives based generally on whether they appear alongside similar recipes in training menus. It uses a statistical graph network that links recipes together based on their co-occurrence in training menus.

At step 1102, process 1100 includes collecting recipes. At step 1104, process 1100 includes collecting menu data, such as training menus that list recipes served together (e.g., together for a particular meal on a particular day).

At step 1106, process 1100 includes constructing a network graph. Process 1100 includes creating a statistical graph network where nodes represent recipes and edges represent co-occurrence of recipes in a meal within a menu. The weight of the edges reflect the frequency the connected recipes appear together in the training menus. This connects recipes that do not occur together but often occur with the same recipes in the meal.

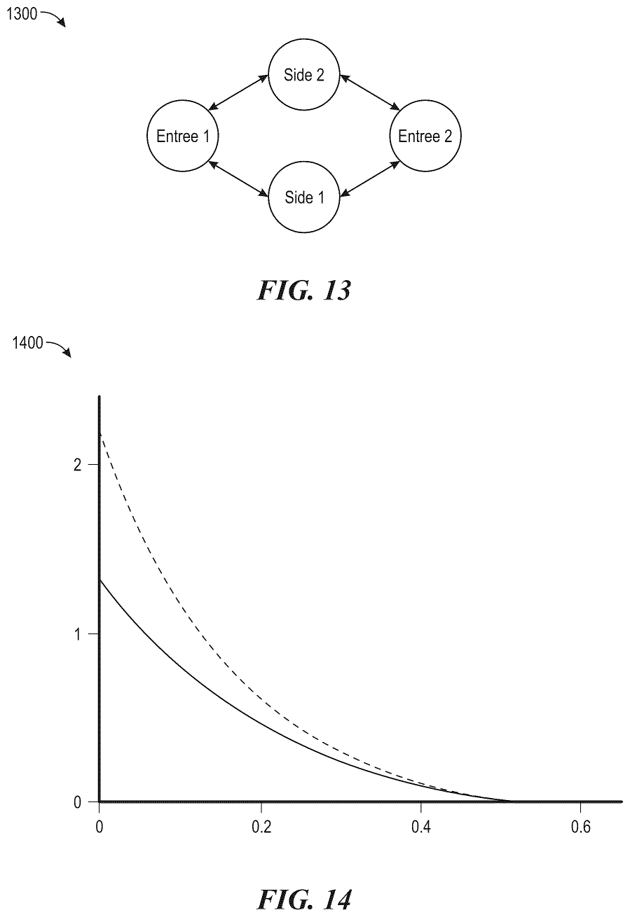

Process 1100 can include receiving a query recipe that potentially requires replacement within a meal. Process 1100 can include identifying the candidate recipes using the constructed graph network based on being 1 hop away (i.e., one intervening node lying between the query recipe and candidate recipe, as illustrated in FIG. 13). Process 1100 can include embedding candidates. For recipes that do not appear in a cycle menu, the recipes can be identified as potential candidates using embeddings based on recipe attributes and description.

At step 1108, process 1100 includes calculating an affinity score (graph weight) pairwise between recipes based on frequency of occurring with the same recipes. Counting the number of edges connecting the recipe candidates through intervening nodes adjusted by the weight of the edges (the weight is based on how often two recipes appear in the same meal of training menus). A pair of recipes will score higher if they frequently appear in a menu with the same other recipes (e.g., burgers and sloppy joes would score highly due to both occurring often (but separately) with coleslaw and potato chips). Process 1100 can include applying a modified sigmoid function to obtain value between 0 and 1. Process 1100 can include applying a threshold to the normalized graph weight to filter out candidates. This could also be a top percentage of candidates, or number of candidates, with a minimum number of candidates. As an illustrative example, a “burger” recipe may be recommended as a good substitute for a “sloppy joe” recipe based on having an above threshold nutritional similarity score (i.e., they are nutritionally equivalent) and above threshold recipe interchangeability (e.g., because both occurring in training menus in meals with “coleslaw”, “potato chips”, etc.). This is even though “burger” and “sloppy joe” may never occur together in the same meal.

FIG. 12 illustrates a diagram of a process 1200 for identifying candidate recipes from a query recipe and from a graph, according to some embodiments of the present technology. At step 1202, process 1200 includes receiving a query recipe that potentially requires replacement within a meal. At step 1204, process 1200 can receive the query recipe and identify candidate recipes using the constructed graph network. At step 1206, process 1200 includes selecting candidate affinity recipes with graph weight.

FIG. 13 illustrates a diagram of an example of graph 1300 showing relationship between two “entrees” connected by two “sides”, according to some embodiments of the present technology.

FIG. 14 illustrates a candidate recipe scoring graph 1400, according to some embodiments of the present technology. The horizontal axis of the graph 1400 is nutrient distance, vertical axis is score, solid line is g=0, dashed line is g=1. The candidate recipe results of the nutritional similarity model and the recipe affinity model can be scored using the below equation (1) to combine each candidate recipes nutritional distance and graph weight based on the query recipe.

$$score = ( 1 + \frac{1 - n}{1 + {e}^{- n}} + {ge}^{- kn} ) ( {e}^{- kn} - m ) \qquad ( 1 )$$

$$where :$$

$$graphWeight = g , 0 <= g <= 1$$

$$nutrientDistance = n , 0 <= n <= 0.5$$

$$k = 4$$

$$midpoint = m = 4$$

The results are filtered by the recipe tags as described below. The identified recommended candidate recipes can then be prioritized based on the calculated PPD for the recipe (or price per serving or portion). Alternatively, the candidate recipes can be prioritized based on other factors or goals, such as improving nutritional balance. For example, a user may indicate a desired minimum or maximum nutritional quantities. Alternatively, candidates may be filtered based on these factors, such as filtering out recipes above a specified PPD, or having one or more nutritional quantities above or below specified thresholds. The prioritizations will determine what the top recipe recommendation will be to optimize a meal for a menu.

Recipes are assigned tags, either manually or using one or more models to classify and tag recipes based on main components. There are four types of tags that are used: meal type tag, recipe type tag, variety tag, and meal pattern tag. Each of these types of tags are then compared with corresponding rules that are used to filter recommended recipes, as described below. The filtering may be done before (or after) calculating the recipe affinity (graph weight) and nutritional similarity scores. Each recipe is assigned one or more meal type tags that identify the type of meal the recipe should be served during (e.g., breakfast, lunch, dinner, snack, etc.). The recommended candidate recipes are filtered to only include recipes with a meal type tag that corresponds with the meal type of the (query) recipe being replaced (i.e., the meal being optimized). This ensures that only recipes that are appropriate for that meal are recommended. Each recipe is assigned one or more recipe type tags that identify its type of food item/recipe (e.g., entree, starchy side, beverage, side salad, etc.). The recommended candidate recipes can be filtered to only include recipes with a recipe type that corresponds with the recipe type of the (query) recipe being replaced (i.e., the meal being optimized). This ensures that only recipes that are appropriate replacements are recommended, e.g., only an entree is recommended for an entree. Each recipe is assigned one or more variety tags that indicate more specific aspects of the recipe. This tag is used to filter out recipes, dynamically based on whether another recipe occurs with the same tag within a set period (e.g., daily rotation, weekly rotation, bi-weekly rotation, etc.). This ensures variety and compliance with dietary guidelines, by preventing specific recipes, food types, or other attributes from being repeated too frequently.

Example rules can include daily rotation, weekly rotation, and bi-weekly rotation. Daily Rotation: Do not repeat the same protein (e.g., tagged with chicken, beef, pork, fish, plant-based protein, etc.) within the same day (or within a 24-hour period). Similarly, do not repeat the same vegetable, fruit, grain, etc. Within the same day or within a 24-hour period. Weekly Rotation: Do not repeat the same type of protein (e.g., chicken, beef, pork, fish, plant-based protein) more than once a week (for each meal type). Similarly, do not repeat the same vegetable, grain, or fruit more than once a week. Bi-Weekly Rotation: Do not repeat the same main dish (tagged) more than once every two-week period. For example, a lasagna recipe should not be repeated for two weeks.

Each recipe is assigned one or more meal pattern tag that indicates the USDA MyPlate Category (e.g., protein, vegetable, fruit, grain, dairy). This tag is used to ensure that each meal has the required variety and balanced components. Recipes candidates that do not maintain a balanced meal are filtered. Each recipe can also be assigned one or more diet type tags that indicate a special diet type that the recipe is appropriate for. This tag can be used to filter recipes by diet type to surface only recipes for recommendation of a particular diet type.

Each recipe can also be assigned a dietary restriction tag that indicates the recipe is not appropriate for a particular dietary restriction or diet types. This tag can be used to filter the recipe out of recommendations when identifying recipes to accommodate residents with corresponding dietary restrictions. The dietary restriction tag can also be used to determine whether an alternate recipe is necessary if one or more residents have a diet type that does not allow for the dietary restriction. Each of the tags can similarly be used to identify recipes in a provided given cycle menu that have potential issues. An alert, warning, indicator, and/or notification can be generated and displayed to a user within a UI (e.g., for a day, meal, and/or recipe on a menu planning calendar user interface). The system can display a recommendation to replace a recipe with another recipe or add a recommended recipe to address this issue.

The identified recommended candidate recipes can be prioritized based on the score calculated above from recipe affinity and nutritional similarity of the candidates. However, the candidate recipes can also be prioritized by the calculated PPD for the recipe (or price per serving). Prioritization can be based on other factors depending on the optimization recommendation, such as improved nutritionals. The prioritizations will determine what the top recipe recommendation will be to optimize a meal for a menu. Prioritization can also include filtering to only the top scoring candidate recipes (e.g., top 2, 3, 4, etc.).

The system may receive up to date inventory levels for a facility and could make recommendations for recipe optimizations to make use of leftover products being used for another recipe. This could be because the minimum purchase quantity for a product may result in significant waste, and by changing another recipe it could make use of the remaining product thereby reducing waste.

In some embodiments, the system may identify when a menu requires an additional recipe to satisfy one or more constraints and may recommend adding an equivalent recipe substitute rather than replacing an existing recipe. For meal pattern regulation, the system may analyze the current meal composition against regulatory requirements such as USDA meal pattern guidelines and determine that an additional recipe is needed to meet minimum component requirements for a particular food group category. For example, if a meal lacks sufficient vegetable servings to comply with meal pattern regulations, the system may recommend adding a vegetable recipe that satisfies the deficiency while maintaining nutritional equivalence with other menu options. For variety checks, the system may detect that a menu violates rotation rules or lacks sufficient variety within a food category and may recommend adding an alternative recipe to provide residents with appropriate choices. For dietary restrictions and allergies, the system may identify that one or more residents cannot consume a recipe in the menu due to allergens or dietary restrictions and may recommend adding an equivalent substitute recipe that accommodates those residents while providing comparable nutritional value and meal pattern contributions. For combo diets, the system may determine that no existing recipe in a meal satisfies the combined constraints of residents assigned to multiple overlapping diet types and may recommend adding a recipe that meets all applicable dietary, nutritional, and regulatory requirements for those residents. The recommended recipe additions may be identified using the nutritional similarity model, recipe affinity model, and tag-based filtering described above, with candidates scored and prioritized based on their ability to address the identified deficiency while maintaining meal balance and cost targets.

The system supports combination diets (“combo diets”), in which a resident or group of residents is subject to multiple diet types and/or dietary restrictions simultaneously. Combo diets may include, for example, combinations of therapeutic diets (e.g., diabetic and renal), dietary restrictions (e.g., low sodium and gluten-free), meal pattern requirements, and regulatory nutrition requirements.

The system may track counts or percentages of residents associated with each diet type, including residents associated with more than one diet type or restriction, and may use this information when determining recipe selections, substitutions, and serving quantities. For example, a resident population may include subsets of residents assigned to overlapping diet categories, and the system may calculate required servings for each subset accordingly. This can be used for determining how much product to order.

Recipes and ingredients may be assigned multiple tags, including diet type tags, dietary restriction tags, meal pattern tags, and nutritional attributes. When evaluating menus for combo diets, the system may apply multiple filtering criteria concurrently to identify recipes that satisfy all applicable constraints, or to identify recipes that violate one or more constraints and therefore require substitution.

In some embodiments, when a recipe does not satisfy the combined requirements of a combo diet, the system may recommend alternative recipes or ingredient substitutions that meet the combined nutritional, dietary, and regulatory requirements. Such recommendations may be based on nutritional similarity, recipe affinity, tag-based filtering, or a combination thereof, and may be prioritized based on one or more optimization goals, including nutritional balance, regulatory compliance, dietary suitability, or cost.

The system may further ensure that recommended recipes for combo diets maintain balanced meals, such as compliance with meal pattern requirements (e.g., USDA MyPlate categories), while also satisfying the multiple diet constraints. Alerts, warnings, or recommendations may be generated when a menu fails to accommodate one or more combo diets, prompting a user to review or accept proposed recipe substitutions or additions.

In some embodiments, the system may generate recommendations to add recipes to a menu to ensure appropriate coverage of therapeutic diets based on resident or patient profiles associated with a facility. The system may analyze the diet type distribution of residents at a facility, including therapeutic diet assignments such as diabetic, renal, cardiac, or dysphagia diets, and compare the current menu offerings against the dietary needs of the resident population. When the system determines that a menu lacks sufficient recipe options for one or more therapeutic diets, the system may recommend adding recipes that satisfy the therapeutic diet requirements while maintaining nutritional equivalence and meal pattern compliance with other menu options. The nutritional similarity model, recipe affinity model, and tag-based filtering described above may be used to identify candidate recipes appropriate for the therapeutic diet, with candidates scored and prioritized based on their suitability for the therapeutic diet, nutritional balance, and cost. Similarly, the same models may be used to identify and recommend ingredient or product swaps within existing recipes to accommodate therapeutic diet requirements. For example, the system may recommend substituting a standard ingredient with a reduced-sodium alternative for residents on a sodium-restricted diet, or substituting a standard bread product with a gluten-free alternative for residents with gluten intolerance. The ingredient or product swap recommendations may be identified by analyzing ingredient tags, nutritional attributes, and product characteristics to find alternatives that satisfy the therapeutic diet constraints while maintaining the overall recipe composition and nutritional profile.

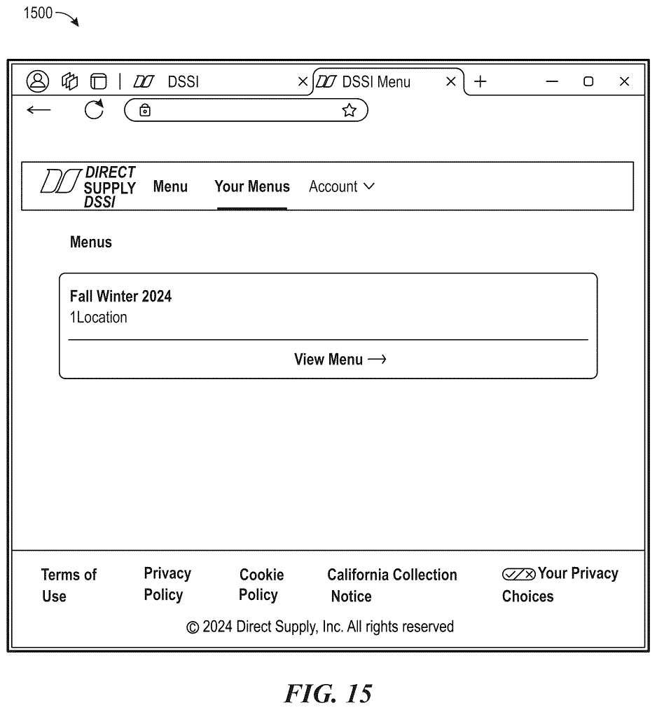

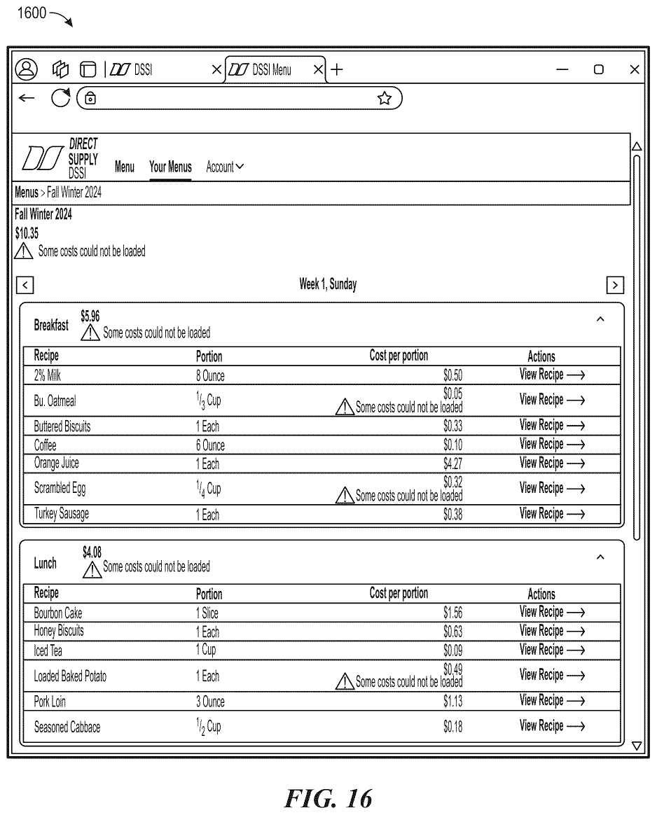

FIGS. 15-18 illustrate examples of menu user interfaces, according to some embodiments of the present technology. The user interfaces 1500, 1600, 1700, and 1800 are provided for managing menus including creating a menu or selecting a menu from a list of existing menus. The menus may be associated with one or more facilities and/or time periods (e.g., month, season, weeks, etc.).

A user interface with a calendar view for displaying and planning a specific menu (i.e., show/plan what is to be served at one or more facilities). The calendar can be shown and planned for a future period of time, such as a month or week. Each day of the menu is shown listing the meals, which can selectively show each of the meal items (i.e., “recipes” as defined above). Each day and meal can be selected and edited to make changes to improve the menu, for example, for savings or nutritional changes. As shown in FIGS. 15-18, each day in the period can be a separate page, or alternatively multiple days can be shown simultaneously on a single page (e.g., week, month, etc.), individual days can then be selected to drill down into the meals and recipes planned for that day.

Recipe recommendations can be highlighted for specific days, meals, or recipes. For example, a user interface element indicator can be displayed to a user indicating an optimization recommendation is identified adjacent a particular recipe (e.g., “optimize”, as shown in FIG. 1700). This indicator can be selected by a user to display the recommendation(s) in more detail for review and selection, for example in a popup modal (as shown in FIG. 18), or on a separate page, in an expandable section, etc. The model may show one or more recommendations each with an element for selecting a corresponding recommended recipe swap; it may also have an element that can be selected to decline the recommendation.

The recommendations provided are to optimize cost-savings and/or to improve nutrition coverage and ultimately help to streamline menu planning and ensure compliance with dietary guidelines. As shown in FIG. 18, each recipe recommendation can display the cost per serving and the positive impact selecting the recommendation will have on cost (e.g., savings per serving or 100 servings; impact on PPD). It may also show the nutrition for each recipe recommendation, and impact on nutrition for the meal (i.e., whether nutritional portion increased/decreased/same). In the menu and/or meal views, an overview of the cost (PPD) and nutritionals can also be displayed for the current meal, day, week, etc. This overview can be shown in the user interface and dependent on the corresponding page being viewed. For example, the user interface can display an overview with average cost (PPD) and nutritionals for a week when viewing a particular week, a day when viewing a particular day, or a meal when viewing a particular day, etc.

A warning icon (e.g., a triangle with exclamation mark) may be shown adjacent to a particular ingredient in the recipe. For example, it may be displayed if a product cannot be sourced because the product is discontinued, stocked out, or otherwise unavailable to one or more locations covered by the menu. Selecting the icon allows the user to manually identify a product or add a product to the order guide. Alternatively, the user can swap out the ingredient or recipe entirely. This can result in a cost being unknown for an ingredient resulting in an inaccurate cost for the portion for the recipe.

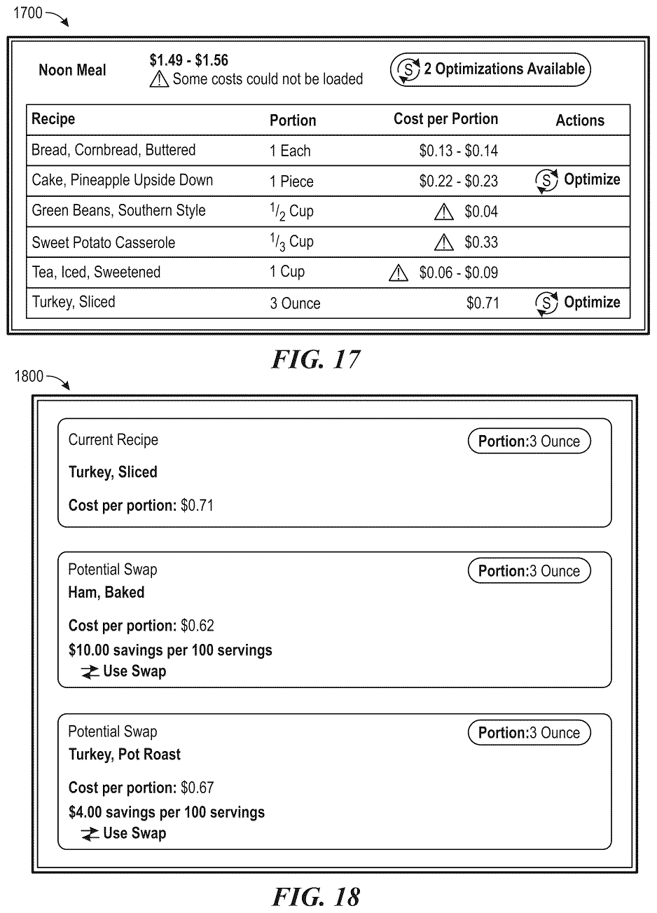

As scan be seen in FIG. 17, ingredient and recipe per portion prices may be shown as a range. This is because not every distribution center will have the same price for same products, and even same products may have different prices at different locations. This range helps to highlight to a corporate menu planning user the price of the recipe even when the price of products varies across facility locations. This is accomplished using our existing order guide management technology.

The UI can show an overview of all selected recommended recipe swaps and may show the total savings, savings PPD, percent savings, etc. Savings may be shown in context of a budget for a specific timeframe (e.g., day, month, year, etc.). The overall nutritional impact of the changes (or lack thereof) can also be shown.

Recipe recommendations can be prioritized for the next best savings opportunity. This can take the form of a list of recommendations that can be addressed, or to determine what recommendations are displayed in the calendar view and which ones are hidden to prevent overwhelming the user and optimize their time spent.

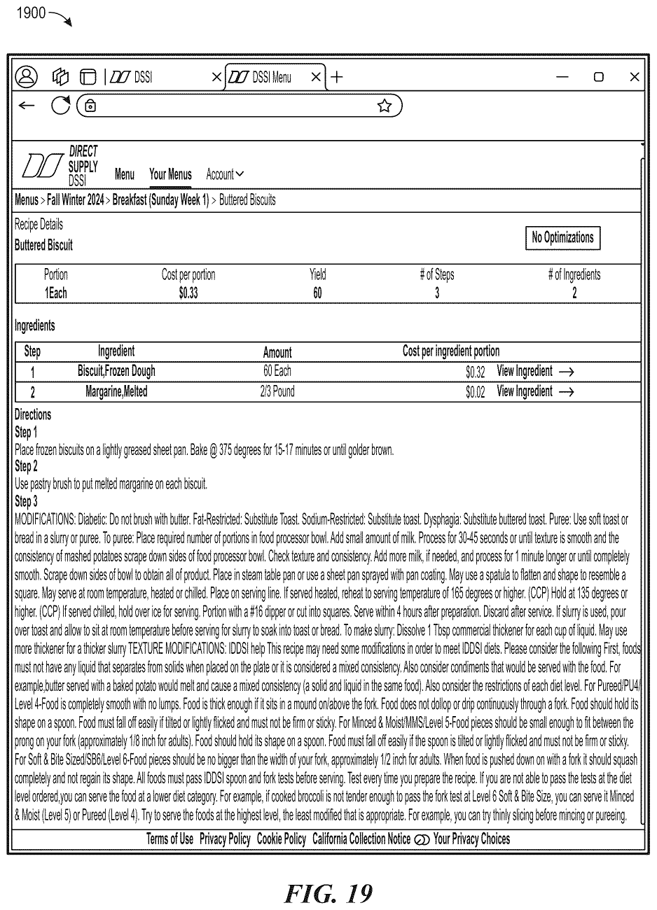

FIG. 19 illustrates an example of a recipe shown in a user interface 1900, according to some embodiments of the present technology. The user interface 1900 has a page for manually editing links between ingredients and products or master item, the unit conversions between products and ingredients, and the nutritional estimates of ingredients.

Page showing ingredients for a particular recipe (e.g., menu>meal>recipe). Provides for manually swapping/overriding the master item assigned to an ingredient. Another page may show all ingredients for the customer or all ingredients for a selected menu, and similar may provide for swapping the master item assigned to each ingredient. Page showing recipe ingredients for a particular recipe (e.g., menu>meal>recipe). Provides for manually altering/overriding the amount of a particular product needed to fill a recipe ingredient. The ingredient page and recipe page can display nutritional estimates for ingredients and recipes, respectively. Provides manual override/editing of the nutritional estimates.

Each recipe has a page showing the portion units, cost per portion, yield of the recipe (i.e., number of portions generated), the number of steps in the recipe, and number of ingredients. It shows a list of ingredients including amount and units, cost per ingredient portion, and an element for navigating to a page for the ingredient. The directions with a detailed description of each step are also displayed. The directions can include additional steps corresponding to dietary restrictions with ways to modify the recipe to comply with certain diets.

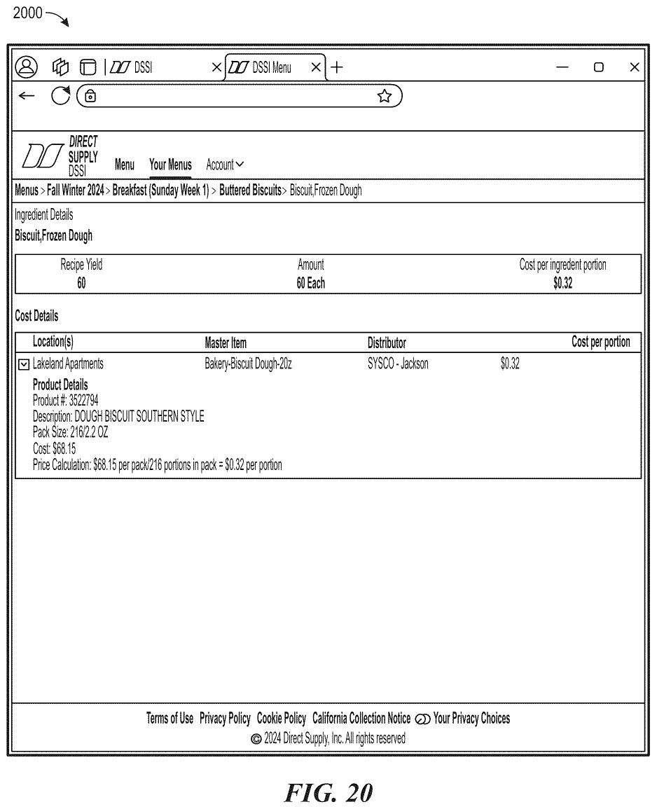

FIG. 20 illustrates an example of individual ingredient details displayed in a user interface 2000, according to some embodiments of the present technology. Each ingredient for a recipe has a page showing the same details shown for the ingredient on the recipe. It also shows the master item, distributor and distribution center. It also shows the linked product details including product number, product name/description, pack size and units, and pack cost, it also shows the cost per portion (as purchased).

Order guides (e.g., formularies) may be synchronized with menu selections. In other words, the products identified for particular ingredients of recipes can be added to an order guide for a customer. This is particularly useful in cases where a recipe requires an ingredient that has no corresponding product on the customer's current order guide. Accordingly, a product can be determined and linked with the ingredient from available products of one or more distributors. Then, when the menu is approved the product can be added to the order guide. It may be added to one or more existing master items or create a new master item. This can require an integration between the menu system and an order guide management system to communicate current order guide and corresponding products and update the order guide to include products.

It is also important to note that a menu may be applied to multiple facilities that are geographically dispersed, such that not all the same products will be available to all facilities (based on availability at corresponding distribution centers). Accordingly, an ingredient for a recipe may be linked to multiple products (e.g., via one or more master items) to ensure coverage of all facilities. This may warrant the addition of a product to an order guide for one facility even if another product for that ingredient already exists but for another facility (see the original OGM 3 patent for further explanation).

Starting with a standard recipe given a therapeutic diet, recipes that need to be substituted can be identified based on nutritionals and tags of recipes in the menu, compared with requirements for a therapeutic diet such as dietary restrictions. Substitute recipes as either an entire recipe or one or more ingredient changes that modify the recipe can be recommended to a user to be added to cover the therapeutic diet residents. The servings required can then be calculated based on the number of residents on the corresponding therapeutic diet. This can then be used to determine the product order quantity as described above by using unit conversion from ingredients to product units.

There is also a way for getting recipe information from PDFs, which includes recipes, ingredients and preparation guide which can be exported from different Menu Systems (Computritions). This could also be through direct integrations. A tool that integrates into browsers and is intended to extract recipes from browser, parse HTML page, and format for saving to DSSI Menu. The tool also has a module for extracting recipes from pictures such as PDF and handwritten notes using OCR, parse, and format to save to DSSI Menu. A recipe generated by an AI tool can also be parsed and reformatted for saving to DSSI Menu. Automatic pricing of menus using real catalogs.

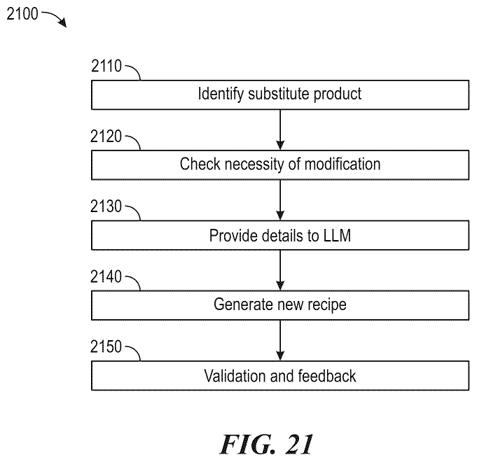

FIG. 21 is a flow diagram illustrating an example process 2100 of updating a recipe, according to some embodiments of the present technology. Process 2100 can include updating recipe steps based on a change to ingredient preparation required for a cheaper substitute product (e.g., canned peaches vs whole peaches) or a substitute product required based on a dietary restriction or requirement. For example, generating a new recipe by modifying an existing recipe.

At step 2110, process 2100 includes identifying a substitute product. The System can find a substitute product that differs substantively from the original product linked to an ingredient in a recipe (e.g., pre-diced peppers for whole peppers). A way this can be done is by finding similar recipes, and then amongst the recipes with ingredient similarity above a threshold. Another way is by directly finding similar ingredients and products that are associated with similar ingredients.

The substitute product can differ substantively from the original product linked to an ingredient in a recipe (e.g., a substitute product of pre-diced peppers for an original product of whole peppers). The substitute product (name/description), original product (name/description), ingredient (name), and list of recipe steps may be provided to a LLM along with a prompt requesting modification of the steps (e.g., by adding, removing, or modifying steps) to accommodate the substitute product in place of the original product, along with one or more examples. The LLM can output the new recipe including a list of steps. Alternatively, it may output only changes to the original recipe, which can be used to modify the original recipe to create the new recipe.

The substitute product may include a product that is substantially the same as the original product but not an equivalent product, such that the substitute product may differ in one or more attributes (e.g., diet type, modifiers, or other tags) while still being suitable as a replacement with the same ingredient. For example, the substitute product may differ based on cost, supplier, dietary suitability (e.g., allergen-free, reduced sodium, or therapeutic diet compliance), or preparation characteristics (i.e., modifiers). In some cases, the substitute product may satisfy a dietary restriction or therapeutic requirement while remaining functionally interchangeable with the original product for purposes of recipe preparation, and therefore may not require changes to the recipe steps. In other cases, the substitute product may differ based on preparation modifiers or form factors (e.g., whole versus pre-diced, raw versus cooked, fresh versus frozen), such that modification or addition of one or more recipe steps is required to accommodate the substitute product. Substitute products may also fall under a same hierarchical level master item.

The substitute product may be identified using one or more similarity-based techniques, including product similarity analysis, ingredient similarity across recipes, or direct association with a common ingredient or ingredient category. In some embodiments, the system identifies candidate substitute products by analyzing recipes having ingredient similarity above a threshold and determining products associated with those recipes, or by identifying alternative products linked to the same ingredient that satisfy additional constraints such as dietary compliance, availability, or pricing. The identified substitute products may then be evaluated to determine whether they are functionally compatible with the original recipe and whether preparation step modifications are necessary.

This process may also be useful when a menu is deployed across multiple facilities (locations) in an organization, where one or more facility may not be able to source a particular product due to distributor coverage, inventory, or contract differences. In such cases, a substitute product may be selected to enable preparation of a facility-specific variation of the same recipe, while maintaining overall menu consistency, nutritional intent, and recipe identity across facilities. The identified substitute products may then be evaluated to determine whether they are functionally compatible with the original recipe and whether preparation step modifications are necessary.

The new recipe with corresponding substitute product may be presented to a dietitian for validation, feedback, or modifications. The updated or validated new recipe may be cached and used for fine tuning the LLM. The process 2100 can include checking that a modification to the recipe steps is necessary based on the identified substitute.

At step 2120, process 2100 includes checking the necessity of a modification. Initially, check if a modification to the recipe steps is necessary based on the identified substitute. This can be done by comparing the substitute product with the original product, to determine if there is a substantive change. This can be done by identifying any “modifiers” (e.g., sliced, whole, etc.) for the products and determining if they differ. This can be done programmatically using a list of known modifiers, and/or by using a LLM to query whether a modification is necessary. This could be incorporated into the prompt of step 2130.

At step 2130, process 2100 includes providing details to an LLM. The system supplies the substitute product information including the product name and description, the original product information including the product name and description, the ingredient name, and the list of recipe steps to a Large Language Model (LLM). The system may also provide additional contextual information such as the form factors of both the substitute product and the original product, the form factor of the original ingredient, and any modifiers associated with the products. Modifiers may include preparation characteristics such as sliced, diced, whole, frozen, or pre-cooked, which can affect how the product is used in the recipe preparation steps.

Process 2100 can include a prompt requesting modification of the steps to accommodate the substitute product. The prompt may instruct the LLM to add new preparation steps when the substitute product requires additional processing that was not needed for the original product, remove existing steps when the substitute product eliminates the need for certain preparation activities, or modify existing steps to reflect changes in timing, technique, or equipment required for the substitute product. For example, if the original product was whole peppers and the substitute product is pre-diced peppers, the prompt may request removal of steps related to washing, coring, and dicing the peppers while potentially adding steps for draining or rinsing the pre-diced product if applicable.

At step 2140, process 2100 includes generating a new recipe. The LLM can output the new recipe, including the complete list of steps required to prepare the recipe using the substitute product. The generated recipe steps may reflect additions, removals, or modifications to the original preparation instructions based on the differences between the substitute product and the original product. For example, if the substitute product is pre-diced vegetables replacing whole vegetables, the LLM may remove steps related to washing, peeling, and dicing while potentially adding steps for draining or rinsing the pre-prepared product.

Alternatively, the LLM may output only the changes to the original recipe rather than the complete new recipe. These changes may be expressed as specific modifications such as step additions, step deletions, or step alterations, which can then be applied to the original recipe to create the new recipe. This approach allows the system to preserve the structure and formatting of the original recipe while incorporating only the necessary adjustments to accommodate the substitute product. The output format may be specified in the prompt to ensure consistent parsing and application of the recipe modifications by the system.

At step 2150, process 2100 includes providing validation and feedback. The system presents the new recipe with the corresponding substitute product to a dietitian or other qualified user for validation, feedback, or modifications. The presentation may include a comparison view showing the original recipe alongside the modified recipe, highlighting the differences in ingredients, preparation steps, and any changes to nutritional values or cost per portion. The dietitian may approve the generated recipe as-is, request additional modifications, or reject the recipe if it does not meet quality standards or dietary requirements. User feedback may be captured through the user interface, including annotations, corrections to specific steps, or alternative ingredient suggestions.

The system caches the updated or validated new recipe for future use and for fine-tuning the LLM. Approved recipes may be stored in a recipe database and associated with the original recipe and substitute product combination, enabling the system to retrieve the validated recipe directly for subsequent requests involving the same substitution scenario without requiring additional LLM processing. Rejected recipes and associated feedback may also be stored and used as negative training examples to improve the accuracy of future recipe generation. Over time, the accumulated validation data from dietitian reviews can be used to fine-tune the LLM, improving its ability to generate accurate and appropriate recipe modifications that require fewer corrections during the validation process.

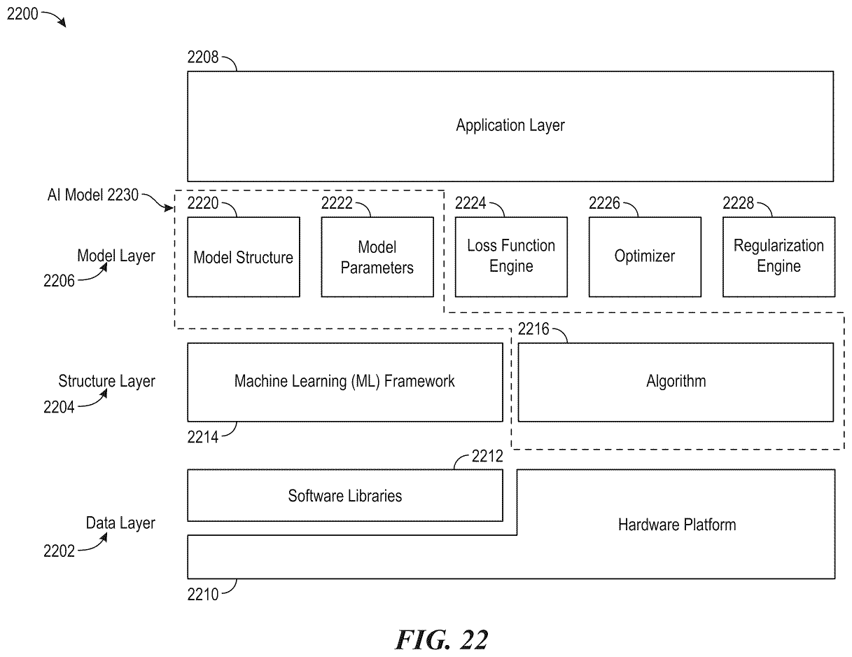

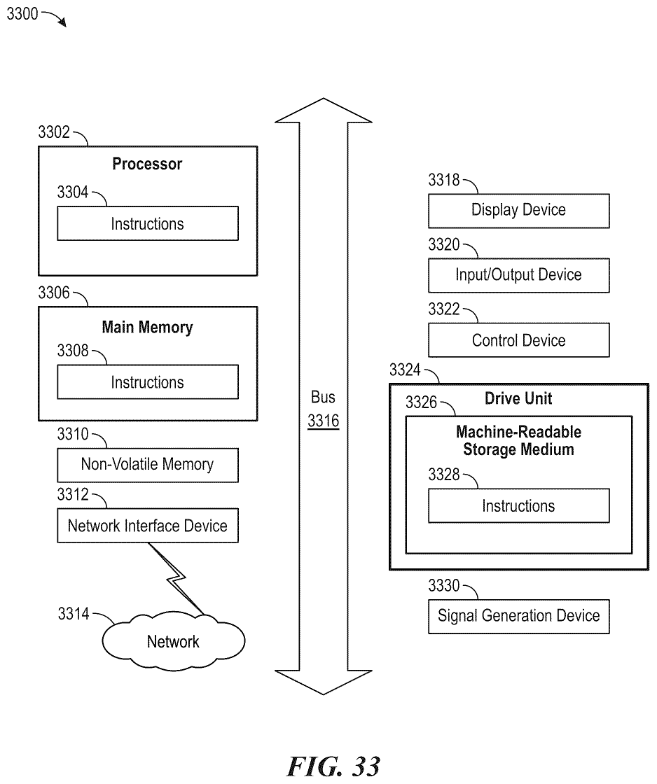

FIG. 22 is a block diagram that illustrates an example AI system that can implement aspects of the present technology. The AI system 2200 is implemented using components of the example computer system 3300 illustrated and described in more detail with reference to FIG. 33. For example, the AI system 2200 can be implemented on the processor 3302 using instructions 3308 programmed in the memory 3306 illustrated and described in more detail with reference to FIG. 33. Likewise, implementations of the AI system 3300 can include different and/or additional components or be connected in different ways.

FIG. 22 illustrates a layered architecture of AI system 2200 that can implement AI models within the system 100 in accordance with some implementations of the present technology. As shown, the AI system 2200 can include a set of layers, which conceptually organize elements within an example network topology for the AI system's architecture to implement a particular AI model 2230. Generally, an AI model 2230 is a computer-executable program implemented by the AI system 2200 that analyses data to make predictions. Information can pass through each layer of the AI system 2200 to generate outputs for the AI model 2230. The layers can include a data layer 2202, a structure layer 2204, a model layer 2206, and an application layer 2208. The algorithm 2216 of the structure layer 2204 and the model structure 2220 and model parameters 2222 of the model layer 2206 together form an example AI model 2230. The optimizer 2226, loss function engine 2224, and regularization engine 2228 work to refine and optimize the AI model 2230, and the data layer 2202 provides resources and support for application of the AI model 2230 by the application layer 2208.

The data layer 2202 acts as the foundation of the AI system 2200 by preparing data for the AI model 2230. As shown, the data layer 2202 can include two sub-layers: a hardware platform 2210 and one or more software libraries 2212. The hardware platform 2210 can be designed to perform operations for the AI model 2230 and include computing resources for storage, memory, logic and networking, such as the resources described in relation to FIG. 33. The hardware platform 2210 can process amounts of data using one or more servers. The servers can perform backend operations such as matrix calculations, parallel calculations, machine learning (ML) training, and the like. Examples of servers used by the hardware platform 2210 include central processing units (CPUs) and graphics processing units (GPUs). CPUs are electronic circuitry designed to execute instructions for computer programs, such as arithmetic, logic, controlling, and input/output (I/O) operations, and can be implemented on integrated circuit (IC) microprocessors. GPUs are electric circuits that were originally designed for graphics manipulation and output but may be used for AI applications due to their vast computing and memory resources. GPUs use a parallel structure that generally makes their processing more efficient than that of CPUs. In some instances, the hardware platform 2210 can include computing resources, (e.g., servers, memory, etc.) offered by a cloud services provider. The hardware platform 2210 can also include computer memory for storing data about the AI model 2230, application of the AI model 2230, and training data for the AI model 2230. The computer memory can be a form of random-access memory (RAM), such as dynamic RAM, static RAM, and non-volatile RAM.

The software libraries 2212 can be thought of suites of data and programming code, including executables, used to control the computing resources of the hardware platform 2210. The programming code can include low-level primitives (e.g., fundamental language elements) that form the foundation of one or more low-level programming languages, such that servers of the hardware platform 2210 can use the low-level primitives to carry out specific operations. The low-level programming languages do not require much, if any, abstraction from a computing resource's instruction set architecture, allowing them to run quickly with a small memory footprint. Examples of software libraries 2212 that can be included in the AI system 2200 include INTEL Math Kernel Library, NVIDIA cuDNN, EIGEN, and OpenBLAS.

The structure layer 2204 can include an ML framework 2214 and an algorithm 2216. The ML framework 2214 can be thought of as an interface, library, or tool that allows users to build and deploy the AI model 2230. The ML framework 2214 can include an open-source library, an application programming interface (API), a gradient-boosting library, an ensemble method, and/or a deep learning toolkit that work with the layers of the AI system facilitate development of the AI model 2230. For example, the ML framework 2214 can distribute processes for application or training of the AI model 2230 across multiple resources in the hardware platform 2210. The ML framework 2214 can also include a set of pre-built components that have the functionality to implement and train the AI model 2230 and allow users to use pre-built functions and classes to construct and train the AI model 2230. Thus, the ML framework 2214 can be used to facilitate data engineering, development, hyperparameter tuning, testing, and training for the AI model 2230. Examples of ML frameworks 2214 that can be used in the AI system 2200 include TENSORFLOW, PYTORCH, SCIKIT-LEARN, KERAS, LightGBM, RANDOM FOREST, and AMAZON WEB SERVICES.

The algorithm 2216 can be an organized set of computer-executable operations used to generate output data from a set of input data and can be described using pseudocode. The algorithm 2216 can include complex code that allows the computing resources to learn from new input data (e.g., temperature measurements and valve positions) and create new/modified outputs based on what was learned. In some implementations, the algorithm 2216 can build the AI model 2230 through being trained while running computing resources of the hardware platform 2210. This training allows the algorithm 2216 to make predictions or decisions without being explicitly programmed to do so. Once trained, the algorithm 2216 can run at the computing resources as part of the AI model 2230 to make predictions or decisions, improve computing resource performance, or perform tasks. The algorithm 2216 can be trained using supervised learning, unsupervised learning, semi-supervised learning, and/or reinforcement learning.

Using supervised learning, the algorithm 2216 can be trained to learn patterns (e.g., map input data to output data) based on labeled training data. The training data may be labeled by an external user or operator. For instance, a user may collect a set of training data, such as by capturing data from sensors, images from a camera, outputs from a model, and the like. In an example implementation, training data can include native-format data collected (e.g., in the form of temperature measurements or valve positions) from various source computing systems described in relation to FIG. 1. Furthermore, training data can include pre-processed data generated by various sensors of the system 100 described in relation to FIG. 1. The user may label the training data based on one or more classes and trains the AI model 2230 by inputting the training data to the algorithm 2216. The algorithm determines how to label the new data based on the labeled training data. The user can facilitate collection, labeling, and/or input via the ML framework 2214. In some instances, the user may convert the training data to a set of feature vectors for input to the algorithm 2216. Once trained, the user can test the algorithm 2216 on new data to determine if the algorithm 2216 is predicting accurate labels for the new data. For example, the user can use cross-validation methods to test the accuracy of the algorithm 2216 and retrain the algorithm 2216 on new training data if the results of the cross-validation are below an accuracy threshold.

Supervised learning can involve classification and/or regression. Classification techniques involve teaching the algorithm 2216 to identify a category of new observations based on training data and are used when input data for the algorithm 2216 is discrete. Said differently, when learning through classification techniques, the algorithm 2216 receives training data labeled with categories (e.g., classes) and determines how features observed in the training data (e.g., valve positions) relate to the categories. Once trained, the algorithm 2216 can categorize new data by analyzing the new data for features that map to the categories. Examples of classification techniques include boosting, decision tree learning, genetic programming, learning vector quantization, k-nearest neighbor (k-NN) algorithm, and statistical classification.

Regression techniques involve estimating relationships between independent and dependent variables and are used when input data to the algorithm 2216 is continuous. Regression techniques can be used to train the algorithm 2216 to predict or forecast relationships between variables. To train the algorithm 2216 using regression techniques, a user can select a regression method for estimating the parameters of the model. The user collects and labels training data that is input to the algorithm 2216 such that the algorithm 2216 is trained to understand the relationship between data features and the dependent variable(s). Once trained, the algorithm 2216 can predict missing historic data or future outcomes based on input data. Examples of regression methods include linear regression, multiple linear regression, logistic regression, regression tree analysis, least squares method, and gradient descent. In an example implementation, regression techniques can be used, for example, to estimate and fill-in missing data for machine-learning based pre-processing operations.

Under unsupervised learning, the algorithm 2216 learns patterns from unlabeled training data. In particular, the algorithm 2216 is trained to learn hidden patterns and insights of input data, which can be used for data exploration or for generating new data. Here, the algorithm 2216 does not have a predefined output, unlike the labels output when the algorithm 2216 is trained using supervised learning. Said another way, unsupervised learning is used to train the algorithm 2216 to find an underlying structure of a set of data, group the data according to similarities, and represent that set of data in a compressed format.

A few techniques can be used in supervised learning: clustering, anomaly detection, and techniques for learning latent variable models. Clustering techniques involve grouping data into different clusters that include similar data, such that other clusters contain dissimilar data. For example, during clustering, data with possible similarities remain in a group that has less or no similarities to another group. Examples of clustering techniques density-based methods, hierarchical based methods, partitioning methods, and grid-based methods. In one example, the algorithm 2216 may be trained to be a k-means clustering algorithm, which partitions _n_ observations in _k_ clusters such that each observation belongs to the cluster with the nearest mean serving as a prototype of the cluster. Anomaly detection techniques are used to detect previously unseen rare objects or events represented in data without prior knowledge of these objects or events. Anomalies can include data that occur rarely in a set, a deviation from other observations, outliers that are inconsistent with the rest of the data, patterns that do not conform to well-defined normal behavior, and the like. When using anomaly detection techniques, the algorithm 2216 may be trained to be an Isolation Forest, local outlier factor (LOF) algorithm, or K-nearest neighbor (k-NN) algorithm. Latent variable techniques involve relating observable variables to a set of latent variables. These techniques assume that the observable variables are the result of training on the latent variables and that the observable variables have nothing in common after controlling for the latent variables. Examples of latent variable techniques that may be used by the algorithm 2216 include factor analysis, item response theory, latent profile analysis, and latent class analysis.

The model layer 2206 implements the AI model 2230 using data from the data layer and the algorithm 2216 and ML framework 2214 from the structure layer 2204, thus enabling decision-making capabilities of the AI system 2200. The model layer 2206 includes a model structure 2220, model parameters 2222, a loss function engine 2224, an optimizer 2226, and a regularization engine 2228.

The model structure 2220 describes the architecture of the AI model 2230 of the AI system 2200. The model structure 2220 defines the complexity of the pattern/relationship that the AI model 2230 expresses. Examples of structures that can be used as the model structure 2220 include decision trees, support vector machines, regression analyses, Bayesian networks, Gaussian processes, genetic algorithms, and artificial neural networks (or, simply, neural networks). The model structure 2220 can include a number of structure layers, a number of nodes (or neurons) at each structure layer, and activation functions of each node. Each node's activation function defines how a node converts data received to data output. The structure layers may include an input layer of nodes that receive input data, an output layer of nodes that produce output data. The model structure 2220 may include one or more hidden layers of nodes between the input and output layers. The model structure 2220 can be an Artificial Neural Network (or, simply, neural network) that connects the nodes in the structured layers such that the nodes are interconnected. Examples of neural networks include Feedforward Neural Networks, convolutional neural networks (CNNs), Recurrent Neural Networks (RNNs), Autoencoder, and Generative Adversarial Networks (GANs).

The model parameters 2222 represent the relationships learned during training and can be used to make predictions and decisions based on input data. The model parameters 2222 can weight and bias the nodes and connections of the model structure 2220. For instance, when the model structure 2220 is a neural network, the model parameters 2222 can weight and bias the nodes in each layer of the neural networks, such that the weights determine the strength of the nodes and the biases determine the thresholds for the activation functions of each node. The model parameters 2222, in conjunction with the activation functions of the nodes, determine how input data is transformed into desired outputs. The model parameters 2222 can be determined and/or altered during training of the algorithm 2216.

The loss function engine 2224 can determine a loss function, which is a metric used to evaluate the AI model's performance during training. For instance, the loss function engine 2224 can measure the difference between a predicted output of the AI model 2230 and the actual output of the AI model 2230 and is used to guide optimization of the AI model 2230 during training to minimize the loss function. The loss function may be presented via the ML framework 2214, such that a user can determine whether to retrain or otherwise alter the algorithm 2216 if the loss function is over a threshold. In some instances, the algorithm 2216 can be retrained automatically if the loss function is greater than the threshold. Examples of loss functions include a binary-cross entropy function, hinge loss function, regression loss function (e.g., mean square error, or quadratic loss), mean absolute error function, smooth mean absolute error function, log-cosh loss function, and quantile loss function.

The optimizer 2226 adjusts the model parameters 2222 to minimize the loss function during training of the algorithm 2216. In other words, the optimizer 2226 uses the loss function generated by the loss function engine 2224 as a guide to determine what model parameters lead to the most accurate AI model. Examples of optimizers include Gradient Descent (GD), Adaptive Gradient Algorithm (AdaGrad), Adaptive Moment Estimation (Adam), Root Mean Square Propagation (RMSprop), Radial Base Function (RBF) and Limited-memory BFGS (L-BFGS). The type of optimizer 2226 used may be determined based on the type of model structure 2220 and the size of data and the computing resources available in the data layer 2202.

The regularization engine 2228 executes regularization operations. Regularization is a technique that prevents over- and under-fitting of the AI model 2230. Overfitting occurs when the algorithm 2216 is overly complex and too adapted to the training data, which can result in poor performance of the AI model 2230. Underfitting occurs when the algorithm 2216 is unable to recognize even basic patterns from the training data such that it cannot perform well on training data or on validation data. The optimizer 2226 can apply one or more regularization techniques to fit the algorithm 2216 to the training data properly, which helps constraint the resulting AI model 2230 and improves its ability for generalized application. Examples of regularization techniques include lasso (L1) regularization, ridge (L2) regularization, and elastic (L1 and L2 regularization). The application layer 2208 describes how the AI system 2200 is used to solve problem or perform tasks.

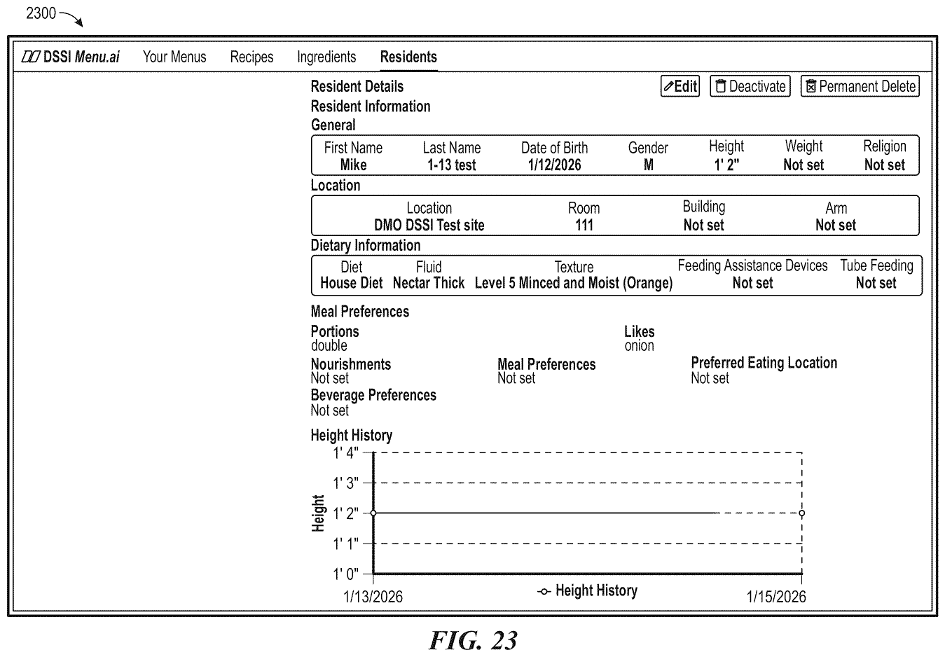

FIG. 23 illustrates a user interface 2300 in which resident data and meal data are used to generate traycards for serving diet and preference compliant meals to residents. The resident data used for traycard generation may be derived from an electronic medical record (EMR) or electronic health record (EHR) system that maintains resident demographic information, diet orders, allergies, and therapeutic diet assignments. Additionally, resident data may be obtained from a dietary management system or resident information database that stores meal preferences, texture modifications, feeding assistance requirements, and other dietary-related attributes specific to each resident. The system can generate individualized meal outputs, such as traycards, for residents or patients based on the planned menu, resident census information, and associated diet types, dietary restrictions, and resident preferences. A traycard represents a per-resident view of resident diet, medical, and preferential information, or may represent a per-resident view of one or more recipes or menu items that may be served to the resident for a particular meal. The traycards may be derived from the same menu and meal planning data used for product ordering and optimization, ensuring consistency between planned menus, dietary coverage, and meal service.

When generating traycards, the system may evaluate each resident's assigned diet type, therapeutic requirements, dietary restrictions, allergies, and preferences, by comparing the resident information against recipes in the planned meal to determine whether the planned menu or meal provides appropriate recipe coverage. If a planned recipe is not suitable for a resident or group of residents, the system may select or recommend an alternate recipe that satisfies the applicable diet constraints and nutritional requirements. The selected or recommended recipe may be substituted for the original recipe on the traycard, added as an additional option, or otherwise reflected in the traycard output to ensure the resident receives a compliant and appropriate meal.

Traycards may be generated automatically for a population of residents, including residents associated with multiple or overlapping diet types, using resident counts or percentages by diet type. The system may aggregate traycard outputs at various levels, such as by facility, meal, diet type, or care unit, and may use the traycard selections to validate menu coverage for therapeutic diets and dietary restrictions. Traycard data may further be used as feedback to identify gaps in menu planning, trigger recipe recommendations, or prompt adjustments to menus or meals to improve dietary coverage, regulatory compliance, or resident satisfaction.

Traycards may be formatted according to one or more predefined or configurable templates, which may specify layout, ordering of information, font sizes, symbols, colors, and other presentation attributes suitable for meal service. The template-based formatting may provide for consistent presentation of resident identifiers, diet indicators, allergens, menu items, and preparation notes, and the templates may be selected based on facility, care unit, meal type, or regulatory or operational requirements. The formatted traycards may be generated in a format suitable for printing, display, or electronic transmission to meal preparation and service staff, while maintaining alignment with the underlying menu, dietary, and recipe selection logic.

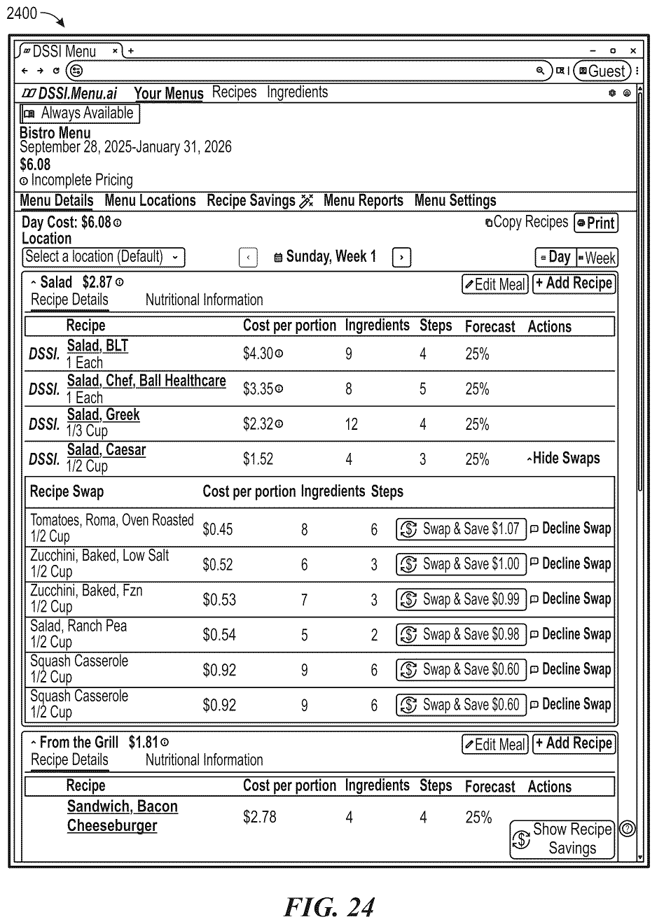

Referring to FIG. 24, a user interface 2400 for recipe swap recommendations is illustrated according to some embodiments of the present technology. The user interface 2400 displays recommendations to swap to a different recipe to capture savings or ensure regulatory compliance. The system surfaces appropriate substitute recipes based on various factors including product availability, order guide compliance, regulatory compliance, nutritional equivalence, and cost optimization. As shown in the user interface 2400, a recipe swap section presents alternative recipe options with associated cost per portion, ingredient counts, and potential savings amounts, allowing users to review and accept or decline recommended swaps. The recommendations may be prioritized based on savings opportunity, with the system calculating and displaying the savings per serving or per specified quantity to help users make informed decisions. In some aspects, the user interface 2400 may display multiple swap options for a single recipe, enabling users to select from several nutritionally equivalent alternatives that satisfy applicable dietary, regulatory, and operational constraints.

Recipes may also be flagged based on product availability, such that a recipe cannot be prepared due to one or more unavailable products, and a substitute recipe may be recommended accordingly. The system may identify when a product linked to a recipe ingredient is discontinued, out of stock, or otherwise unavailable at one or more distribution centers covering the facility. In such cases, the system may recommend a temporary substitute recipe that can be prepared with available products, or may recommend a substitute unique to a particular location where a particular product is unavailable. This location-specific substitution allows the menu to maintain consistency across facilities while accommodating variations in product availability due to distributor coverage, inventory levels, or contract differences. The user interface 2400 may display indicators identifying recipes with availability issues and may present recommended substitutes that satisfy the same nutritional, regulatory, and meal pattern requirements as the original recipe while using products that are available for the affected facility or facilities.

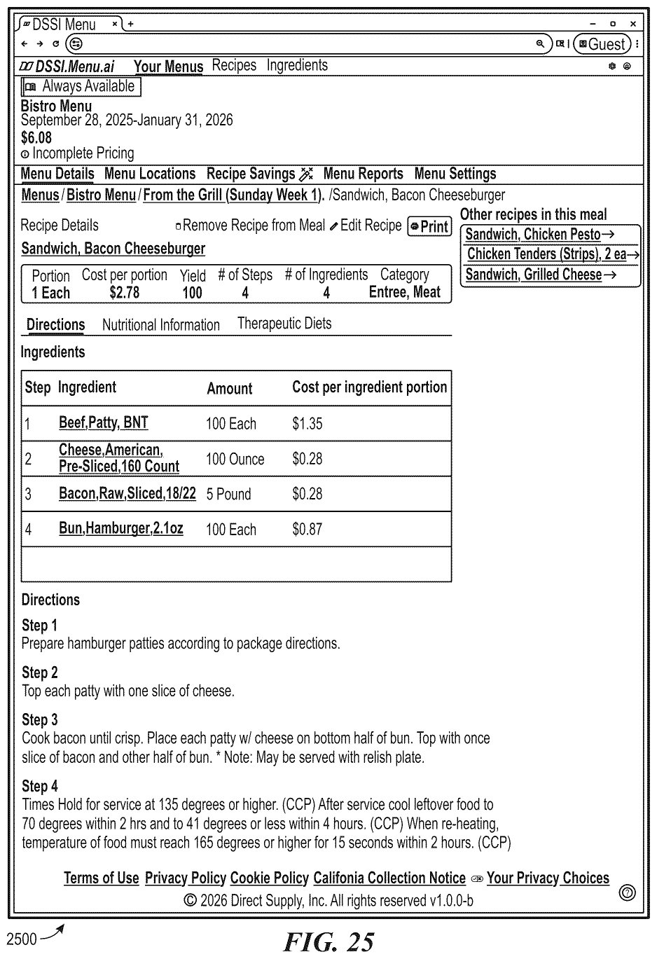

Referring to FIG. 25, a user interface 2500 for updated recipe detail views is illustrated according to some embodiments of the present technology. The user interface 2500 may display recipe information including portion size, cost per portion, yield, number of steps, number of ingredients, and recipe category, along with a detailed ingredients table showing step numbers, amounts, and costs per ingredient portion. The user interface 2500 also may present preparation directions with numbered steps and may include links to other recipes in the same meal, as well as options to remove, edit, or print the recipe.

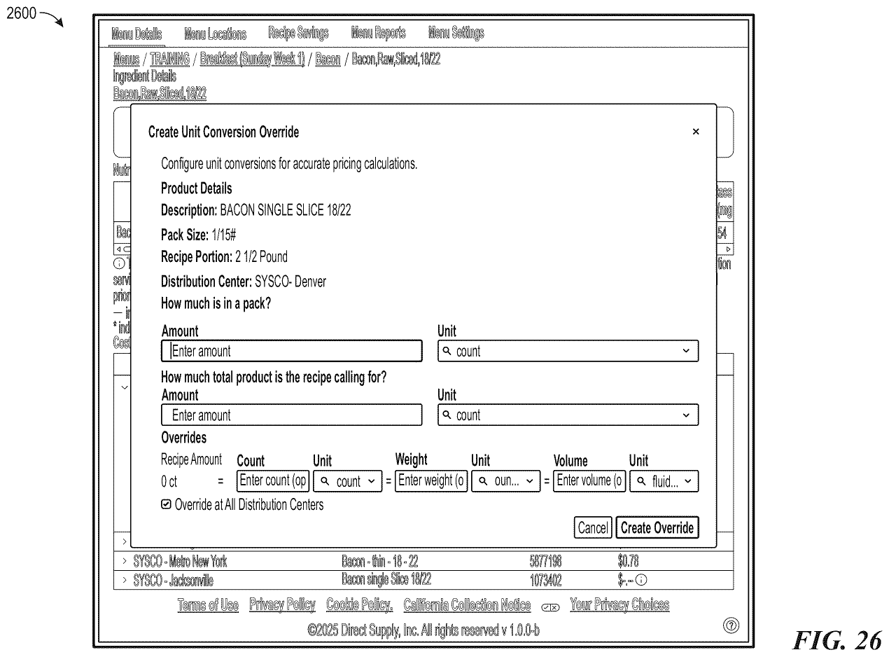

Referring to FIG. 26, a user interface 2600 for overriding product unit conversions is illustrated according to some embodiments of the present technology. The user interface 2600 provides a modal for creating unit conversion overrides by allowing users to specify how much product is in a pack and how much total product the recipe requires, with fields for entering amounts and selecting units such as count, weight, or volume. The user interface 2600 may include an option to apply the override at all distribution centers or at specific distribution centers, and displays location-specific product information for review.

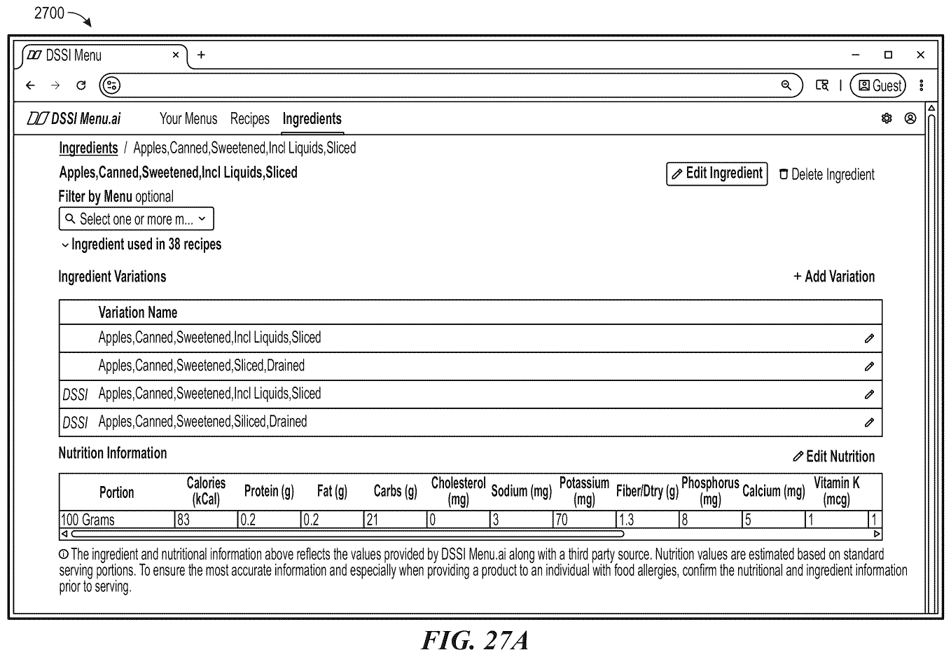

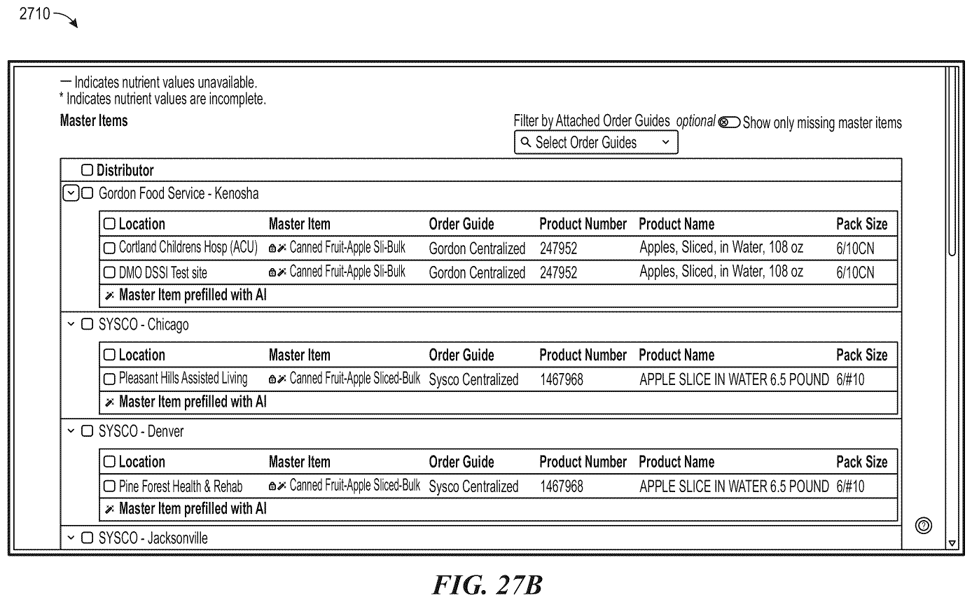

Referring to FIG. 27A, a user interface 2700 for an enhanced ingredient view is illustrated according to some embodiments of the present technology. The user interface 2700 displays ingredient details including ingredient variations, associated product information, and nutritional data for a specified portion size. The user interface 2700 may provide options to edit the ingredient, delete the ingredient, or add variations, and includes a disclaimer regarding the source of nutritional information.

Referring to FIG. 27B, a user interface 2710 for an enhanced ingredient view with master item information is illustrated according to some embodiments of the present technology. The user interface 2710 may display master item associations organized by distributor and location, showing the linked master item, order guide, product number, product name, and pack size for each facility. The user interface 2710 includes filtering options to show only missing master items or filter by attached order guides, and indicates when master items have been prefilled using AI.

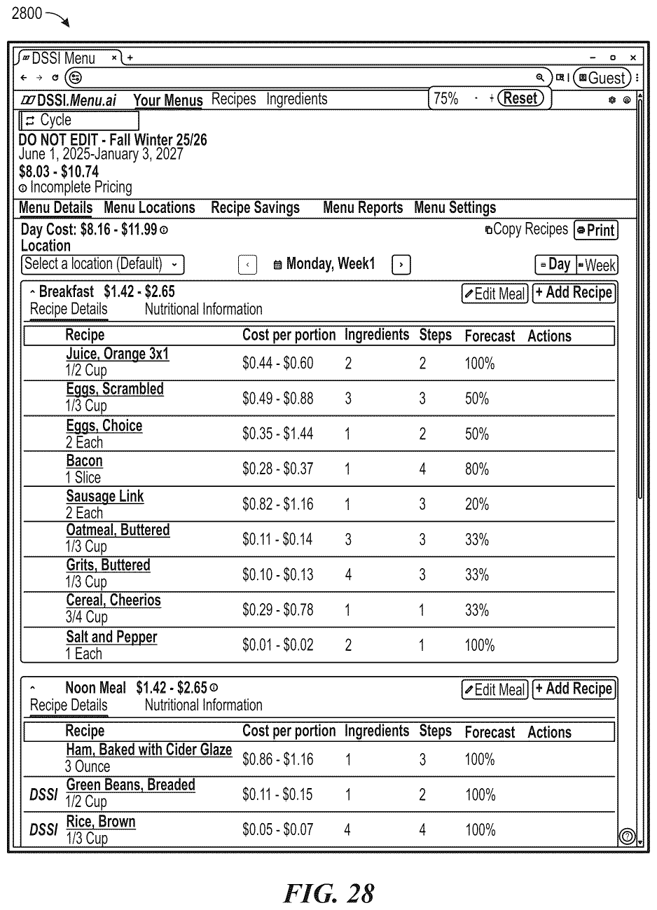

Referring to FIG. 28, a user interface 2800 for setting individual recipe forecast percentage is illustrated according to some embodiments of the present technology. The user interface 2800 may display a menu with recipes organized by meal period, allowing users to set forecast percentages for each individual recipe within a meal. The forecast percentages may be used in calculating the quantity of products needed for ordering based on anticipated resident selections.

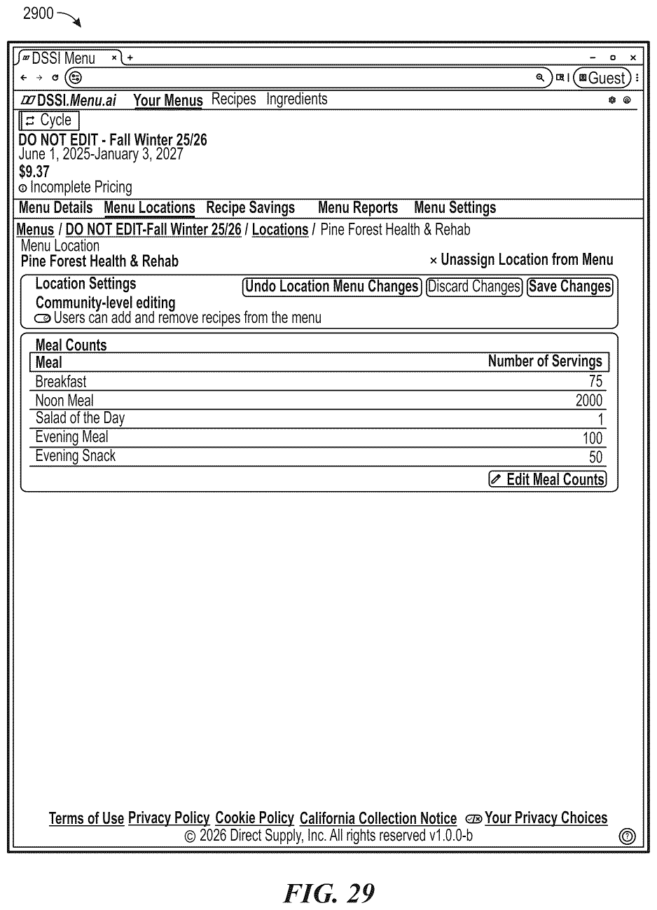

Referring to FIG. 29, a user interface 2900 for setting servings per meal at a location level is illustrated according to some embodiments of the present technology. The user interface 2900 may display location-specific menu settings including a meal counts table that lists each meal period and the corresponding number of servings configured for that location. The user interface 2900 provides options to edit meal counts and configure community-level editing permissions for adding and removing recipes from the menu.

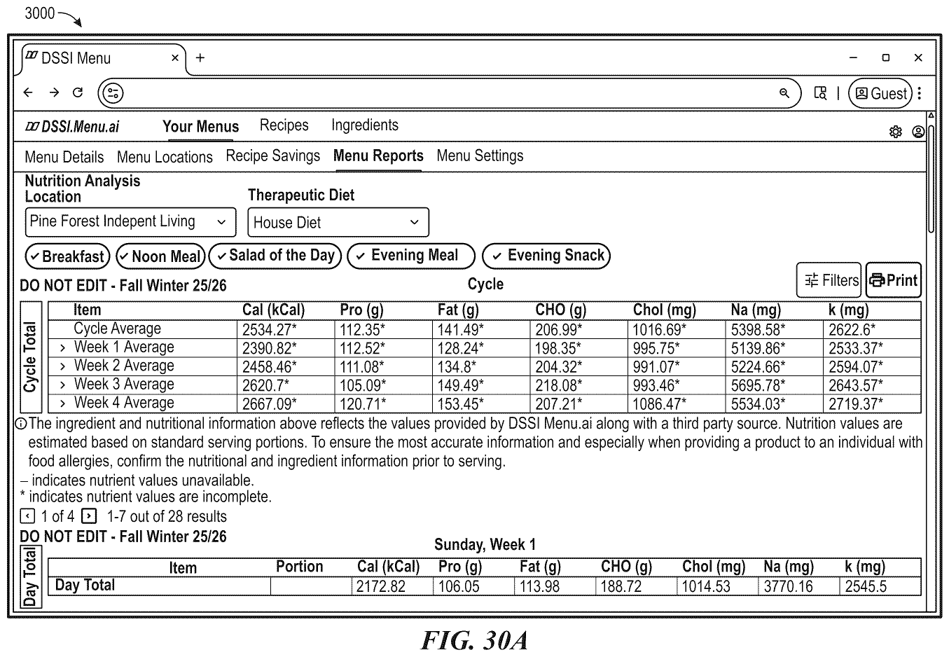

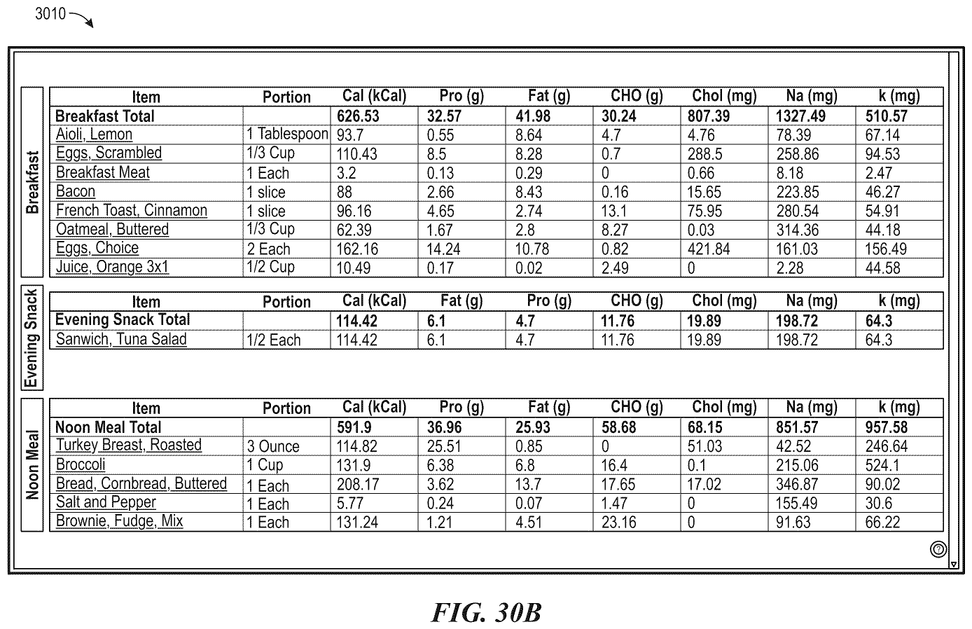

Referring to FIG. 30A, a user interface 3000 for nutritional analysis is illustrated according to some embodiments of the present technology. The user interface 3000 may display nutritional information for a menu cycle, including cycle averages and weekly averages for nutrients such as calories, protein, fat, carbohydrates, cholesterol, sodium, and potassium. The user interface 3000 allows filtering by location, therapeutic diet, and meal period, and displays day totals for selected dates.

Referring to FIG. 30B, a user interface 3010 for nutritional analysis with meal period details is illustrated according to some embodiments of the present technology. The user interface 3010 may display nutritional information organized by meal period, with tables showing individual recipe items and their corresponding nutritional values per portion. Each meal period table may include a total row summarizing the aggregate nutritional values for that meal.

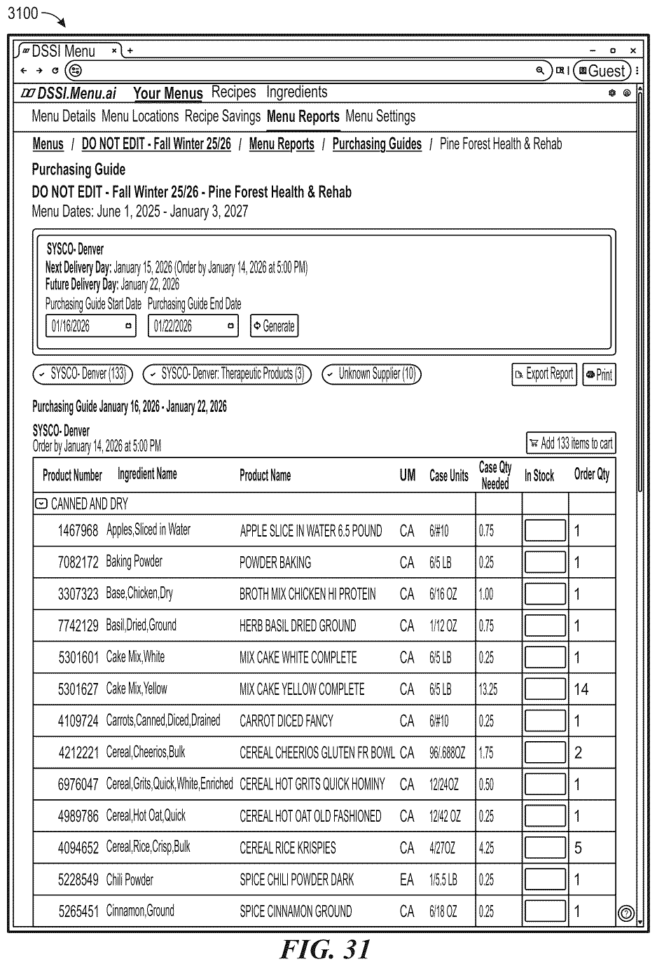

Referring to FIG. 31, a user interface 3100 for a purchasing workflow is illustrated according to some embodiments of the present technology. The user interface 3100 may display a purchasing guide generated from menu ingredients for a specified order period, showing supplier information, delivery dates, and order deadlines. The user interface 3100 presents a product listing table with columns for product details, case quantities needed, in-stock quantities, and order quantities, allowing users to review and adjust orders before adding items to a procurement cart.

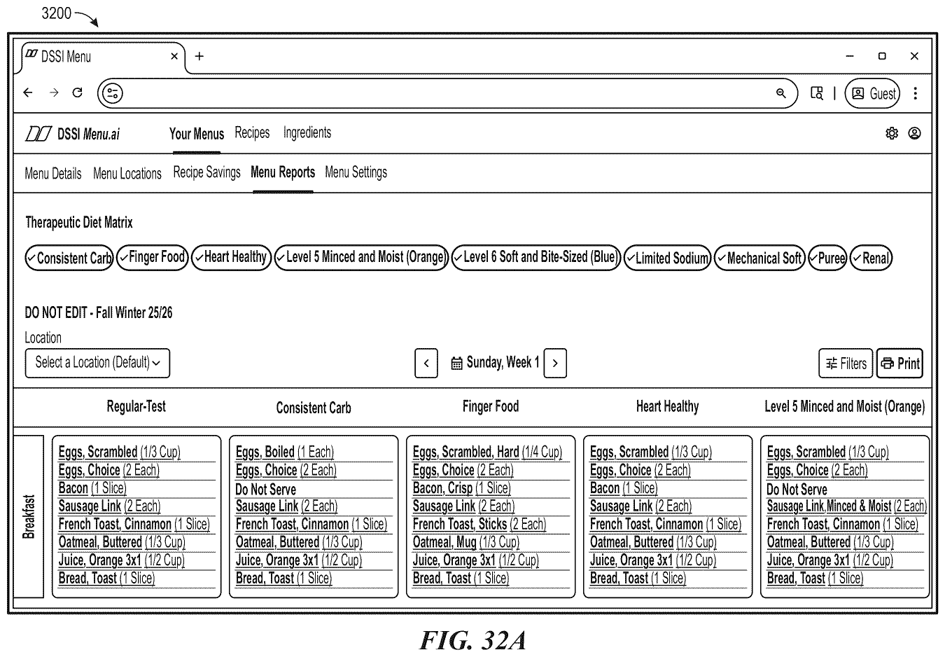

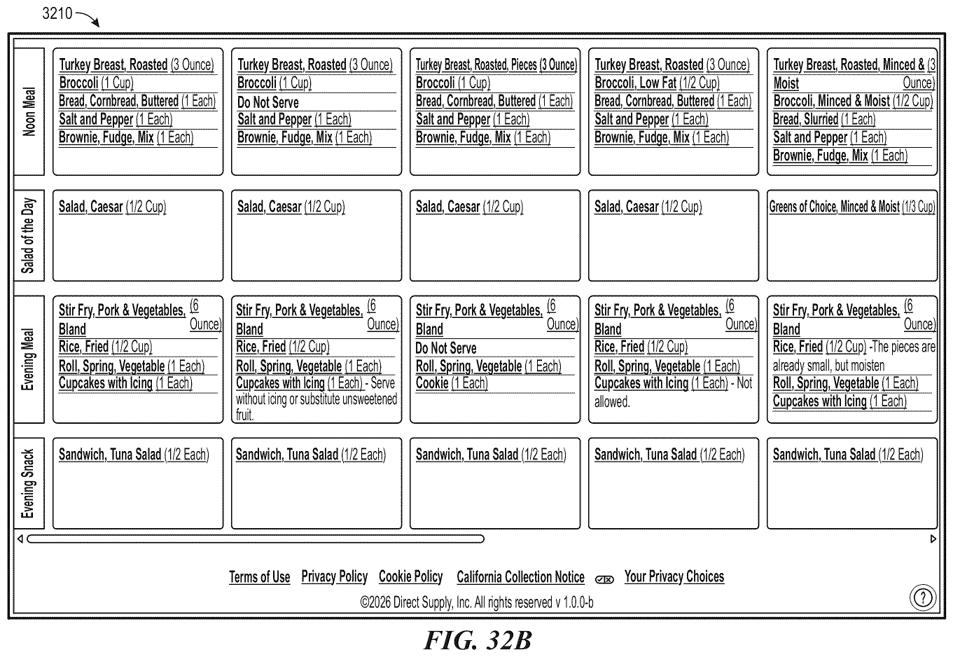

Referring to FIG. 32A, a user interface 3200 for menu views across diet types is illustrated according to some embodiments of the present technology. The user interface 3200 may display a therapeutic diet matrix showing recipe variations across multiple diet type columns, allowing users to view how recipes are adapted for different dietary requirements. The user interface 3200 includes indicators for recipe modifications and do-not-serve designations where a recipe is not appropriate for a particular diet type.

Referring to FIG. 32B, a user interface 3210 for menu views across diet types with modification indicators is illustrated according to some embodiments of the present technology. The user interface 3210 may display a therapeutic diet matrix showing recipe variations across multiple diet type columns, allowing users to view how recipes are adapted for different dietary requirements. The user interface 3210 may show modification indicators such as do-not-serve notations and substitution instructions for recipes that require adaptation for specific therapeutic diets. In some aspects, the user interface 3210 may present recipe substitute or addition recommendations when the system identifies that a menu lacks sufficient coverage for one or more therapeutic diets based on resident population requirements. These recommendations may be surfaced alongside the therapeutic diet matrix to enable users to address diet type coverage deficiencies by accepting suggested recipe substitutions or additions that satisfy the applicable dietary, nutritional, and regulatory constraints.

FIG. 33 is a block diagram that illustrates an example of a computer system in which at least some operations described herein can be implemented. Components of the computer system 3300 can be used to implement the system 150 or 300B.

As shown, the computer system 3300 can include: one or more processors 3302, main memory 3306, non-volatile memory 3310, a network interface device 3312, video display device 3318, an input/output device 3320, a control device 3322 (e.g., keyboard and pointing device), a drive unit 3324 that includes a storage medium 3326, and a signal generation device 3330 that are communicatively connected to a bus 3316. The bus 3316 represents one or more physical buses and/or point-to-point connections that are connected by appropriate bridges, adapters, or controllers. Various common components (e.g., cache memory) are omitted from FIG. 33 for brevity. Instead, the computer system 3300 is intended to illustrate a hardware device on which components illustrated or described relative to the examples of the figures and any other components described in this specification can be implemented.

The computer system 3300 can take any suitable physical form. For example, the computer system 3300 can share a similar architecture as that of a server computer, personal computer (PC), tablet computer, mobile telephone, game console, music player, wearable electronic device, network-connected (“smart”) device (e.g., a television or home assistant device), AR/VR systems (e.g., head-mounted display), or any electronic device capable of executing a set of instructions that specify action(s) to be taken by the computer system 3300. In some implementation, the computer system 3300 can be an embedded computer system, a system-on-chip (SOC), a single-board computer system (SBC) or a distributed system such as a mesh of computer systems or include one or more cloud components in one or more networks. Where appropriate, one or more computer systems 3300 can perform operations in real-time, near real-time, or in batch mode.

The network interface device 3312 enables the computer system 3300 to mediate data in a network 3314 with an entity that is external to the computer system 3300 through any communication protocol supported by the computer system 3300 and the external entity. Examples of the network interface device 3312 include a network adaptor card, a wireless network interface card, a router, an access point, a wireless router, a switch, a multilayer switch, a protocol converter, a gateway, a bridge, bridge router, a hub, a digital media receiver, and/or a repeater, as well as all wireless elements noted herein.

The memory (e.g., main memory 3306, non-volatile memory 3310, machine-readable medium 3326) can be local, remote, or distributed. Although shown as a single medium, the machine-readable medium 3326 can include multiple media (e.g., a centralized/distributed database and/or associated caches and servers) that store one or more sets of instructions 3328. The machine-readable (storage) medium 3326 can include any medium that is capable of storing, encoding, or carrying a set of instructions for execution by the computer system 3300. The machine-readable medium 3326 can be non-transitory or include a non-transitory device. In this context, a non-transitory storage medium can include a device that is tangible, meaning that the device has a concrete physical form, although the device can change its physical state. Thus, for example, non-transitory refers to a device remaining tangible despite this change in state.

Although implementations have been described in the context of fully functioning computing devices, the various examples are capable of being distributed as a program product in a variety of forms. Examples of machine-readable storage media, machine-readable media, or computer-readable media include recordable-type media such as volatile and non-volatile memory devices 3310, removable flash memory, hard disk drives, optical disks, and transmission-type media such as digital and analog communication links.

In general, the routines executed to implement examples herein can be implemented as part of an operating system or a specific application, component, program, object, module, or sequence of instructions (collectively referred to as “computer programs”). The computer programs typically include one or more instructions (e.g., instructions 3304, 3308, 3328) set at various times in various memory and storage devices in computing device(s). When read and executed by the processor 3302, the instruction(s) cause the computer system 3300 to perform operations to execute elements involving the various aspects of the disclosure.

The embodiments, features, systems, devices, materials, methods and techniques described herein may, in some embodiments, be similar to any one or more of the embodiments, features, systems, devices, materials, methods and techniques described in the following: U.S. Patent Application Pub. No. 2021/0241881, U.S. Patent Application Pub. No. 2020/0272968, U.S. Pat. Nos. 12,106,842, and 10,685,308 which are incorporated by reference in their entireties.

The above description and drawings are illustrative and are not to be construed as limiting. Numerous specific details are described to provide a thorough understanding of the disclosure. However, in some instances, well-known details are not described in order to avoid obscuring the description. Further, various modifications may be made without deviating from the scope of the embodiments.

Reference in this specification to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

The terms used in this specification generally have their ordinary meanings in the art, within the context of the disclosure, and in the specific context where each term is used. It will be appreciated that the same thing can be said in more than one way. Consequently, alternative language and synonyms may be used for any one or more of the terms discussed herein, and any special significance is not to be placed upon whether or not a term is elaborated or discussed herein. Synonyms for some terms are provided. A recital of one or more synonyms does not exclude the use of other synonyms. The use of examples anywhere in this specification, including examples of any term discussed herein, is illustrative only and is not intended to further limit the scope and meaning of the disclosure or of any exemplified term. Likewise, the disclosure is not limited to various embodiments given in this specification. Unless otherwise defined, all technical and scientific terms used herein have the same meaning as commonly understood by one of ordinary skill in the art to which this disclosure pertains. In the case of conflict, the present document, including definitions, will control.

What is claimed is:

1. A method for linking ingredients to products and performing unit conversions in a management system, the method comprising:
receiving a query ingredient associated with a recipe stored in a database, wherein the query ingredient includes an ingredient name and one or more recipe ingredient units specifying an amount and unit of measure required by the recipe;
generating, using an embedding model comprising a neural network, a query embedding vector for the query ingredient based on the ingredient name;
retrieving, from a product database, a plurality of master items, wherein each master item is associated with one or more products available from a distribution center;
generating, using the embedding model, one or more master item embedding vectors for the plurality of master items based on master item names and product descriptions;
selecting a master item from the plurality of master items based on a similarity between the query embedding vector and the one or more master item embedding vectors;
determining a unit conversion between product units of a product associated with the selected master item and recipe ingredient units of the query ingredient by querying a conversion database and applying one or more conversion factors; and
displaying, via a graphical user interface, the recipe including the query ingredient, the selected master item, the product associated with the selected master item, and a cost per portion for the query ingredient.

2. The method of claim 1, further comprising:
retrieving resident profile data from a resident information database, wherein the resident profile data includes a resident diet type one or more dietary restrictions, and one or more resident preferences;
comparing the resident profile data to one or more recipes in a planned meal to determine whether each recipe satisfies the resident diet type and the one or more dietary restrictions;
in response to determining that the one or more recipes in the planned meal do not satisfy the resident diet type, or the one or more dietary restrictions, selecting an alternate recipe; and
generating a traycard that includes the resident diet type, the one or more dietary restrictions, and the alternate recipe substituted for the recipe.

3. The method of claim 1, further comprising:
analyzing a menu to determine whether the menu satisfies one or more constraints associated with a facility;
in response to determining that the menu does not satisfy the one or more constraints, identifying a deficiency in the menu;
identifying, using at least one of a nutritional similarity model, a recipe affinity model, or tag-based filtering, a candidate recipe that addresses the deficiency; and
recommending, via the graphical user interface, adding the candidate recipe to the menu as an equivalent recipe substitute to address the deficiency.

4. The method of claim 1, further comprising:
identifying a substitute product for the product associated with the selected master item by analyzing one or more recipes having ingredient similarity above a threshold and determining one or more products associated with the one or more recipes;
evaluating the substitute product to determine whether the substitute product satisfies one or more constraints including at least one of dietary compliance, product availability at the distribution center, or pricing requirements; and
in response to determining that the substitute product satisfies the one or more constraints, linking the substitute product to the query ingredient as an alternative to the product associated with the selected master item.

5. The method of claim 1, further comprising:
identifying, based on at least one of a cost analysis or a regulatory compliance analysis, one or more alternative recipes that are nutritionally equivalent to the recipe;
determining a savings amount associated with swapping the recipe to each of the one or more alternative recipes based on a cost per portion comparison;
displaying, via the graphical user interface, a recipe swap recommendation including the one or more alternative recipes, wherein each alternative recipe is presented with an associated cost per portion, a savings amount, and an indication of regulatory compliance status; and
responsive to receiving a user selection of an alternative recipe from the recipe swap recommendation, substituting the alternative recipe for the recipe in a menu.

6. The method of claim 1, further comprising:
determining a form factor of the product units and a form factor of the recipe ingredient units;
in response to determining that the form factor of the product units matches the form factor of the recipe ingredient units, applying a direct conversion from a conversion table; and
in response to determining that the form factor of the product units does not match the form factor of the recipe ingredient units, querying the conversion database using an embedding search to identify an indirect conversion based on density information associated with the query ingredient.

7. The method of claim 1, further comprising:
calculating a similarity score for each master item based on a distance between the query embedding vector and a corresponding master item embedding vector;
applying a threshold similarity score to filter the plurality of master items to obtain a list of candidate master items;
providing the list of candidate master items to a large language model to select a match; and
receiving, from the large language model, a selection of the master item from the list of candidate master items.

8. A system comprising:
one or more processors; and
one or more memories storing instructions that, when executed by the one or more processors, cause the system to perform a process for linking ingredients to products and performing unit conversions, the process comprising:
receiving a query ingredient associated with a recipe stored in a database, wherein the query ingredient includes an ingredient name and one or more recipe ingredient units specifying an amount and unit of measure required by the recipe;
generating, using an embedding model comprising a neural network, a query embedding vector for the query ingredient based on the ingredient name;
retrieving, from a product database, a plurality of master items, wherein each master item is associated with one or more products available from a distribution center;
generating, using the embedding model, one or more master item embedding vectors for the plurality of master items based on master item names and product descriptions;
selecting a master item from the plurality of master items based on a similarity between the query embedding vector and the one or more master item embedding vectors;
determining a unit conversion between product units of a product associated with the selected master item and recipe ingredient units of the query ingredient by querying a conversion database and applying one or more conversion factors; and
displaying, via a graphical user interface, the recipe including the query ingredient, the selected master item, the product associated with the selected master item, and a cost per portion for the query ingredient.

9. The system of claim 8, wherein the process further comprises:
retrieving resident profile data from a resident information database, wherein the resident profile data includes a resident diet type one or more dietary restrictions, and one or more resident preferences;
comparing the resident profile data to one or more recipes in a planned meal to determine whether each recipe satisfies the resident diet type and the one or more dietary restrictions;
in response to determining that the one or more recipes in the planned meal do not satisfy the resident diet type, or the one or more dietary restrictions, selecting an alternate recipe; and
generating a traycard that includes the resident diet type, the one or more dietary restrictions, and the alternate recipe substituted for the recipe.

10. The system of claim 8, wherein the process further comprises:
analyzing a menu to determine whether the menu satisfies one or more constraints associated with a facility;
in response to determining that the menu does not satisfy the one or more constraints, identifying a deficiency in the menu;
identifying, using at least one of a nutritional similarity model, a recipe affinity model, or tag-based filtering, a candidate recipe that addresses the deficiency; and
recommending, via the graphical user interface, adding the candidate recipe to the menu as an equivalent recipe substitute to address the deficiency.

11. The system of claim 8, wherein the process further comprises:
identifying a substitute product for the product associated with the selected master item by analyzing one or more recipes having ingredient similarity above a threshold and determining one or more products associated with the one or more recipes;
evaluating the substitute product to determine whether the substitute product satisfies one or more constraints including at least one of dietary compliance, product availability at the distribution center, or pricing requirements; and
in response to determining that the substitute product satisfies the one or more constraints, linking the substitute product to the query ingredient as an alternative to the product associated with the selected master item.

12. The system of claim 8, wherein the process further comprises:
identifying, based on at least one of a cost analysis or a regulatory compliance analysis, one or more alternative recipes that are nutritionally equivalent to the recipe;
determining a savings amount associated with swapping the recipe to each of the one or more alternative recipes based on a cost per portion comparison;
displaying, via the graphical user interface, a recipe swap recommendation including the one or more alternative recipes, wherein each alternative recipe is presented with an associated cost per portion, a savings amount, and an indication of regulatory compliance status; and
responsive to receiving a user selection of an alternative recipe from the recipe swap recommendation, substituting the alternative recipe for the recipe in a menu.

13. The system of claim 8, wherein the process further comprises:
determining a form factor of the product units and a form factor of the recipe ingredient units;
in response to determining that the form factor of the product units matches the form factor of the recipe ingredient units, applying a direct conversion from a conversion table; and
in response to determining that the form factor of the product units does not match the form factor of the recipe ingredient units, querying the conversion database using an embedding search to identify an indirect conversion based on density information associated with the query ingredient.

14. The system of claim 8, wherein the process further comprises:
calculating a similarity score for each master item based on a distance between the query embedding vector and a corresponding master item embedding vector;
applying a threshold similarity score to filter the plurality of master items to obtain a list of candidate master items;
providing the list of candidate master items to a large language model to select a match; and
receiving, from the large language model, a selection of the master item from the list of candidate master items.

15. A non-transitory computer-readable medium storing instructions that, when executed by a computing system, cause the computing system to perform operations for linking ingredients to products and performing unit conversions, the operations comprising:
receiving a query ingredient associated with a recipe stored in a database, wherein the query ingredient includes an ingredient name and one or more recipe ingredient units specifying an amount and unit of measure required by the recipe;
generating, using an embedding model comprising a neural network, a query embedding vector for the query ingredient based on the ingredient name;
retrieving, from a product database, a plurality of master items, wherein each master item is associated with one or more products available from a distribution center;
generating, using the embedding model, one or more master item embedding vectors for the plurality of master items based on master item names and product descriptions;
selecting a master item from the plurality of master items based on a similarity between the query embedding vector and the one or more master item embedding vectors;
determining a unit conversion between product units of a product associated with the selected master item and recipe ingredient units of the query ingredient by querying a conversion database and applying one or more conversion factors; and
displaying, via a graphical user interface, the recipe including the query ingredient, the selected master item, the product associated with the selected master item, and a cost per portion for the query ingredient.

16. The non-transitory computer-readable medium of claim 15, wherein the operations further comprise:
retrieving resident profile data from a resident information database, wherein the resident profile data includes a resident diet type one or more dietary restrictions, and one or more resident preferences;
comparing the resident profile data to one or more recipes in a planned meal to determine whether each recipe satisfies the resident diet type and the one or more dietary restrictions;
in response to determining that the one or more recipes in the planned meal do not satisfy the resident diet type, or the one or more dietary restrictions, selecting an alternate recipe; and
generating a traycard that includes the resident diet type, the one or more dietary restrictions, and the alternate recipe substituted for the recipe.

17. The non-transitory computer-readable medium of claim 15, wherein the operations further comprise:
analyzing a menu to determine whether the menu satisfies one or more constraints associated with a facility;
in response to determining that the menu does not satisfy the one or more constraints, identifying a deficiency in the menu;
identifying, using at least one of a nutritional similarity model, a recipe affinity model, or tag-based filtering, a candidate recipe that addresses the deficiency; and
recommending, via the graphical user interface, adding the candidate recipe to the menu as an equivalent recipe substitute to address the deficiency.

18. The non-transitory computer-readable medium of claim 15, wherein the operations further comprise:
identifying a substitute product for the product associated with the selected master item by analyzing one or more recipes having ingredient similarity above a threshold and determining one or more products associated with the one or more recipes;
evaluating the substitute product to determine whether the substitute product satisfies one or more constraints including at least one of dietary compliance, product availability at the distribution center, or pricing requirements; and
in response to determining that the substitute product satisfies the one or more constraints, linking the substitute product to the query ingredient as an alternative to the product associated with the selected master item.

19. The non-transitory computer-readable medium of claim 15, wherein the operations further comprise:
identifying, based on at least one of a cost analysis or a regulatory compliance analysis, one or more alternative recipes that are nutritionally equivalent to the recipe;
determining a savings amount associated with swapping the recipe to each of the one or more alternative recipes based on a cost per portion comparison;
displaying, via the graphical user interface, a recipe swap recommendation including the one or more alternative recipes, wherein each alternative recipe is presented with an associated cost per portion, a savings amount, and an indication of regulatory compliance status; and
responsive to receiving a user selection of an alternative recipe from the recipe swap recommendation, substituting the alternative recipe for the recipe in a menu.

20. The non-transitory computer-readable medium of claim 15, wherein the operations further comprise:
determining a form factor of the product units and a form factor of the recipe ingredient units;
in response to determining that the form factor of the product units matches the form factor of the recipe ingredient units, applying a direct conversion from a conversion table; and
in response to determining that the form factor of the product units does not match the form factor of the recipe ingredient units, querying the conversion database using an embedding search to identify an indirect conversion based on density information associated with the query ingredient.
